(19) United States
(12) Patent Application Publication
(10) Pub. No.: US 2006/0160138 A1
(43) Pub. Date: Jul. 20, 2006

(54) Compositions and methods for protein design

(76) Inventors: George Church, Brookline, MA (US); Brian M. Baynes, Cambridge, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 11/332,657

(22) Filed: Jan. 13, 2006

Related U.S. Application Data
(60) Provisional application No. 60/643,813, filed on Jan. 13, 2005.

(51) Int. Cl. G01N 33/53; G06F 19/00

(52) U.S. Cl. 435/7.1; 702/19

(57) ABSTRACT

In certain aspects the present invention provides methods and compositions related to rational protein design.

### RELATED APPLICATIONS

This application claims the benefit of priority to U.S. Provisional Application No. 60/643,813, filed Jan. 13, 2005, which application is hereby by incorporated by reference in its entirety.

### BACKGROUND

Directed molecular evolution can be used to create proteins such as enzymes with novel functions and properties. Starting with a known natural protein, several rounds of mutagenesis, functional screening, and propagation of successful sequences are performed. The advantage of this process is that it can be used to rapidly evolve any protein without knowledge of its structure. Several different mutagenesis strategies exist, including point mutagenesis by error-prone PCR, cassette mutagenesis, and DNA shuffling. These techniques have had many successes; however, they are all handicapped by their inability to produce more than a tiny fraction of the potential changes. For example, there are 20500 possible amino acid changes for an average protein approximately 500 amino acids long. Clearly, the mutagenesis and functional screening of so many mutants is impossible; directed evolution provides a very sparse sampling of the possible sequences and hence examines only a small portion of possible improved proteins, typically point mutants or recombinations of existing sequences. By sampling randomly from the vast number of possible sequences, directed evolution is unbiased and broadly applicable, but inherently inefficient because it ignores all structural and biophysical knowledge of proteins.

In contrast, computational methods can be used to screen enormous sequence libraries (up to 10⁸⁰ in a single calculation) overcoming the key limitation of experimental library screening methods such as directed molecular evolution. There are a wide variety of methods known for generating and evaluating sequences. These include, but are not limited to, sequence profiling (Bowie and Eisenberg, Science 253(5016): 164-70, (1991)), rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)); and residue pair potentials (Jones, Protein Science 3: 567-574, (1994)).

Computational methods are powerful methods as an initial step to identify potential protein variants that may exhibit a desired characteristic. However, it is still necessary to experimentally test a number, typically a large number of variants to determine if they do indeed exhibit the predicited characteristic or property. Currently, methodologies for obtaining large numbers of purposefully diverse molecular species have prevented practioners from screening large numbers of variants identified using in silico methods. There is a great need for new compositions and methods that will permit high throughput experimental confirmation of in silico predictions.

Furthermore, a technique for the manufactue of truly diverse candidate structures which themselves could be further mutagenized as necessary would be a very effective way to explore DNA, RNA and protein structure space. Such a technique would enable production of a family of designs embodying “rational diversity,” providing tens, hundreds, or multiple thousands of different constructs embodying, for example, multiple, evolutionarily independent design approaches adapted for selection, screening, or random combinatorial, further rational mutagenesis. This would permit the discovery of DNA, protein and cellular constructs that are evolutionarily unlikely to be obtained, and permit the protein engineer to traverse and explore rugged fitness space. Stated differently, the availability of such techniques would permit avoidance of “Darwin's black box,” the logical difficulty of reaching through evolution a particular biological state where all intermediate preceding states are evolutionarily disadvantaged, lethal, or require simultaneous alterations of biochemistry or structure very unlikely to occur. It is an object of the present invention to provide methods for rational protein design, including high throughput methods for producing and experimentally evaluating large numbers of variants identified using computational methods.

### SUMMARY OF THE INVENTION

The present invention provides compositions and methods for designing a protein having a desired characteristic.

In one aspect, the invention provides a biosynthetic library comprising a plurality of synthetic DNAs of known and planned, as opposed to randomized, sequence. These encode a plurality of candidate proteins which can be selected or screened for species having a predetermined property or set of properties, or may be selected or screened themselves for polynucleotides having particular functional or structural properties, e.g., ribosomal activity. The polynucleotides in the libraries preferably are chemically synthesized or are assembled from chemically synthesized oligonucleotides using techniques such as set forth herein. The plural DNAs they contain may comprise regions of significant sequence homology. Alternatively, or in addition, the library members have reading frames exploiting consistent codon usage patterns so as to promote similar expression levels in a selected cellular or cell free expression system, e.g., a ribosomal expression system, a phage expression system, or an E coli expression system. Preferably, the oligonucleotides are synthesized in parallel. It is also preferred to assemble the genes in parallel from the chemically synthesized oligonucleotides.

In another aspect, the invention provides a method for producing a protein having a desired characteristic comprising:
i) applying an algorithm to a protein scaffold to generate a plurality of possible variants;
ii) screening the plurality of variants in silico to produce a rank ordered list of variants;
iii) generating nucleic acid molecules having predefined sequences that encode at least 10 of the variants wherein the nucleic acid molecules are generated by a method comprising:
   a) providing a pool of oligonucleotides comprising partially overlapping sequences that define the sequence of each of said nucleic acid molecules that encode said variants;
   b) incubating said pool of oligonucleotides under hybridization conditions and at least one of the following conditions: (i) ligation conditions, (2) chain extension conditions, or (iii) chain extension and ligation conditions, thereby forming nucleic acid constructs; and
   c) separating constructs having said predefined sequences from constructs not having said predefined sequences, thereby forming the nucleic acid molecules that encode said variants; and
iv) causing expression of said nucleic acid molecules to produce said protein variants; and
v) screening the variants to identify variants having the desired characteristic.

In certain embodiments, the methods may be used to produce nucleic acids encoding at least 100, 1000, 10,000, or more, of the variants.

In certain embodiments, the nucleic acids enclosing the variants are each at least 1000, 5000, or more, bases in length.

In certain embodiments, the methods may further comprise inserting the nucleic acids encoding the variants into a plasmid, such as, for example, an expression plasmid. The methods may further comprise introducing the nucleic acids encoding the variants, or introducing a plasmid comprising the nucleic acids encoding the variants, into a cell. In certain embodiments, the variants may be produced in a cell, such as, for example, a bacterial cell. In other embodiments, the variants may be produced in vitro. In certain embodiments, the nucleic acid molecules encoding the variants may comprise a regulatory sequence, such as, for example, a promoter or an enhancer.

In certain embodiments, at least a portion of the nucleic acid molecules encoding the variants are prepared in a single pool. In other embodiments, all, or a substantial portion, of the nucleic acid molecules encoding the variants are prepared in a single pool.

In certain embodiments, at least a portion of the sequence of one or more nucleic acids encoding the variants has been codon remapped to reduce the homology with at least one other nucleic acid.

In certain embodiments, the variants may be screened to identify a variant having at least one of the following characteristics: an enzymatic activity, a structural feature, a binding affinity for a target molecule, improved stability, lower immunogenicity, better bioavailability, increased expression, or increased solubility.

In certain embodiments, the oligonucleotides are synthesized on an array. In certain such embodiments, the array may comprise a solid support and a plurality of discrete features associated with said solid support, wherein each feature independently comprises a population of oligonucleotides collectively having a defined consensus sequence but in which no more than 10 percent of said oligonucleotides of said feature have the identical sequence. In certain embodiments, the method for generating the nucleic acid molecules further comprises an error reduction process.

In certain embodiments, the nucleic acid molecules encoding the variants comprise sticky ends.

In certain embodiments, one or more of the oligonucleotides that define the sequence of the nucleic acid molecules further comprises sequence tags such that a set of oligonucleotides that defines the sequence of a nucleic acid construct having a desired sequence has a distinguishable complement of sequence tags as compared to a set of oligonucleotides that defines the sequence of an incorrect product, and wherein nucleic acid constructs having a desired sequence are separated from incorrect crossover products based on size or electrophoretic mobility.

In certain embodiments, a set of oligonucleotides that defines the sequence of a nucleic acid construct having a desired sequence forms sticky ends that permit circularization of the correctly formed product, and wherein correctly formed circularized products are separated from incorrectly formed linear products. In such embodiments, the circularized products may be separated from the linear products by digesting the linear products with an exonuclease or by size separation, for example, using gel electrophoresis.

In certain embodiments, the nucleic acid molecules encoding the variants comprise vector sequences and sticky ends that permit circularization of the nucleic acid molecule to produce a circularized expression plasmid.

In another aspect, the invention provides a biosynthetic library comprising a plurality of synthetic DNAs encoding a plurality of candidate proteins which can be selected or screened for species having a predetermined property or set of properties, the library comprising plural DNAs comprising regions of sequence homology and being assembled from chemically synthesized oligonucleotides. In certain embodiments, the chemically synthesized oligonucleotides are synthesized in parallel. In certain embodiments, the DNAs are assembled in parallel from chemically synthesized oligonucleotides.

In another aspect, the invention provides a biosynthetic library comprising a plurality of synthetic DNAs encoding a plurality of candidate proteins which can be selected or screened for species having a predetermined property or set of properties, the library comprising plural DNAs chemically synthesized or assembled from chemically synthesized oligonucleotides and comprising reading frames of multiple said DNAs exploiting consistent codon usage patterns so as to promote similar expression levels in a selected expression system. In certain embodiments, the chemically synthesized oligonucleotides are synthesized in parallel. In certain embodiments, the DNAs are assembled in parallel from chemically synthesized oligonucleotides.

In another aspect, the invention provides a biosynthetic library comprising at least 10 DNAs of pre specified, purposefully generated sequence chemically synthesized or assembled from chemically synthesized oligonucleotides and encoding a plurality of candidate proteins which can be selected or screened for species having a predetermined property or set of properties. In certain embodiments, the chemically synthesized oligonucleotides are synthesized in parallel. In certain embodiments, the DNAs are assembled in parallel from chemically synthesized oligonucleotides.

In another aspect, the invention provides a method for producing a protein having a desired characteristic or property comprising generating sequence data for a plurality of possible protein variants; generating plural oligonucleotides in parallel and assembling them to produce nucleic acid molecules that encode at least 10 of the sequences of the protein variants; expressing the nucleic acid molecules to produce the protein variants; and selecting or screening the variants to identify proteins having the desired characteristic. In certain embodiments, the method involves assembling the oligonucleotides by hybridization of complementary oligonucleotide sequences followed by ligase and/or polymerase treatment, and produces at least 20, 50, 100, 10³, 10⁴, 10⁵, or 10⁶ of the sequences of the protein variants.

In certain embodiments, the methods provided herein may involve generating a library of scaffold protein variants that may be rank-ordered to identify variant sequences of particular interest. A large number of the protein variants may then be expressed and experimentally tested to identify variants that exhibit the desired characteristic. The methods involve construction of large nucleic molecules with high fidelity using stepwise assembly of complementary, overlapping, oligonucleotides. In exemplary embodiments, at least 10, 100, 1,000, 10,000, 100,000 or more protein variants are experimentally tested.

The practice of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

The claims provided below are hereby incorporated into this section by reference.

### BRIEF DESCRIPTION OF THE FIGURES

FIG. 1 illustrates three exemplary methods for assembly of construction oligonucleotides into subassemblies and/or polynucleotide constructs, including (A) ligation, (B) chain extension, and (C) chain extension plus ligation. The dotted lines represent strands that have been extended by polymerase.

FIG. 2 shows a simplified illustration of an example DNA molecule to be synthesized.

FIG. 3 illustrates a microarray used in the synthesis of the exemplary DNA molecule of FIG. 1.

FIG. 4 illustrates possible crossover products that may arise when conducting multiplex assembly of polynucleotide constructs with internal homologous regions.

FIG. 5 illustrates crossover polymerization that may occur when conducting multiplex assembly of polynucleotide constructs with internal homologous regions.

FIG. 6 illustrates one embodiment of the circle selection method for multiplex assembly of polynucleotide constructs containing regions of homology.

FIG. 7 illustrates another embodiment of the circle selection method for multiplex assembly of polynucleotide constructs containing regions of homology.

FIG. 8 illustrates one embodiment of the size selection method for multiplex assembly of polynucleotide constructs containing regions of homology.

FIG. 9 illustrates another embodiment of the size selection method for multiplex assembly of polynucleotide constructs containing regions of homology.

FIG. 10 illustrates a method for removal of error sequences using mismatch binding proteins.

FIG. 11 illustrates a method for neutralization of error sequences with mismatch recognition proteins.

FIG. 12 illustrates a method for strand-specific error correction.

FIG. 13 shows one scheme for local removal of DNA on both strands at the site of a mismatch.

FIG. 14 shows another scheme for local removal of DNA on both strands at the site of a mismatch.

FIG. 15 summarizes the effects of the methods of FIG. 13 (or equivalently, FIG. 14) applied to two DNA duplexes, each containing a single base (mismatch) error.

FIG. 16 shows an example of semi-selective removal of mismatch-containing segments.

FIG. 17 shows a procedure for reducing correlated errors in synthesized DNA.

### DETAILED DESCRIPTION OF THE INVENTION

1. Definitions

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

The term “amplification” means that the number of copies of a nucleic acid fragment is increased.

The term “characteristic,” as used herein with reference to a protein or protein variant, refers to a biochemical and/or biophysical property of a protein. Examples of biophysical properties, include for example, thermal stability, solubility, isoelectric point, pH stability, crystalizability, conditions of crystallization, aggregation state, heat capacity, resistance to chemical denaturation, resistance to proteolytic degradation, amide hydrogen exchange data, behavior on chromatographic matrices, electrophoretic mobility, resistance to degradation during mass spectrometry, and results obtained from nuclear magnetic resonance, X-ray crystallography, circular dichroism, light scattering, atomic adsorption, fluorescence, fluorescence quenching, mass spectroscopy, infrared spectroscopy, electron microscopy, and/or atomic force microscopy. Examples of biochemical properties include, for example, expressability, protein yield, small-molecule binding, subcellular localization, utility as a drug target, protein-protein interactions, and protein-ligand interactions.

The term “cleavage” as used herein refers to the breakage of a bond between two nucleotides, such as a phosphodiester bond.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

“Domain” refers to a unit of a protein or protein complex, comprising a polypeptide subsequence, a complete polypeptide sequence, or a plurality of polypeptide sequences where that unit has a defined function. The function is understood to be broadly defined and can be ligand binding, catalytic activity or can have a stabilizing effect on the structure of the protein.

The term “gene” refers to a nucleic acid comprising an open reading frame encoding a polypeptide having exon sequences and optionally intron sequences. The term “intron” refers to a DNA sequence present in a given gene which is not translated into protein and is generally found between exons.

The term “heterologous” as used herein in the context of a chimeric polynucleotide, refers to sequences comprising segments, domains, or genetic elements, the exact combination and sequence of which is not found in nature.

The term “ligase” refers to a class of enzymes and their functions in forming a phosphodiester bond in adjacent oligonucleotides which are annealed to the same oligonucleotide. Particularly efficient ligation takes place when the terminal phosphate of one oligonucleotide and the terminal hydroxyl group of an adjacent second oligonucleotide are annealed together across from their complementary sequences within a double helix, i.e. where the ligation process ligates a “nick” at a ligatable nick site and creates a complementary duplex (Blackburn, M. and Gait, M. (1996) in Nucleic Acids in Chemistry and Biology, Oxford University Press, Oxford, pp. 132-33, 481-2). The site between the adjacent oligonucleotides is referred to as the “ligatable nick site”, “nick site”, or “nick”, whereby the phosphodiester bond is non-existent, or cleaved.

The term “ligate” refers to the reaction of covalentlyjoining adjacent oligonucleotides through formation of an internucleotide linkage.

The term “motif” refers to an amino acid sequence that is commonly found in a protein of a particular structure or function. Typically, a consensus sequence is defined to represent a particular motif. The consensus sequence need not be strictly defined and may contain positions of variability, degeneracy, variability of length, etc. The consensus sequence may be used to search a database to identify other proteins that may have a similar structure or function due to the presence of the motif in its amino acid sequence. For example, on-line databases may be searched with a consensus sequence in order to identify other proteins containing a particular motif. Various search algorithms and/or programs may be used, including FASTA, BLAST or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md.

The term “mutations” means changes in the sequence of a wild-type nucleic acid sequence or changes in the sequence of a wild-type polypeptide sequence. Such mutations may be point mutations such as transitions or transversions. The mutations may be deletions, insertions or duplications.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

“Polypeptide” and “peptide” are used interchangeably herein to refer to a polymer of amino acid residues; whereas a “protein” typically contains one or multiple polypeptide chains. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “residue” as it relates to a polynucleotide or polypeptide refers to either a purine or pyrimidine nucleotide for polynucleotides, or an amino acid for a polypeptide.

The term “structural motif”, when used in reference to a polypeptide, refers to a polypeptide that, although it may have different amino acid sequences, may result in a similar structure, wherein by structure is meant that the motif forms generally the same tertiary structure, or that certain amino acid residues within the motif, or alternatively their backbone or side chains (which may or may not include the CaL atoms of the side chains) are positioned in a like relationship with respect to one another in the motif.

The term “wild-type” means that the nucleic acid fragment does not comprise any mutations. A “wild-type” protein means that the protein will be active at a comparable level of activity found in nature and typically will comprise the amino acid sequence found in nature. In an aspect of the invention, the term “wild type” or “parental sequence” can indicate a starting or reference sequence prior to a manipulation of the sequence.

2. Protein Engineerin Using Rational Diversity

De novo protein design methodologies have become significantly more powerful in the past decade. It is now possible to screen libraries of >10¹⁰⁰ protein sequences in silico, not by computationally checking each one, but rather by exploiting an algorithm to eliminate regions of sequence space. See Design of a Novel Globular Protein Fold with Atomic Level Accuracy, Kuhlman et al., Science, V203, p. 1344, 2003. These library sizes are staggering in comparison with experimental methods, which top out at library sizes of about 10¹² to 10¹⁵.

The caveat of in silico methods is that they rely heavily on empirical models of protein function, and thus, currently have far less than perfect accuracy. To compensate for model inaccuracies, the output of in silico models is generally a rank-ordered list of possible designs, where each design is assigned a score. One then ends up with a list of “highly likely solutions” at the top of this ordered list, some subset of which can be synthesized or mutated from wild type sequences and tested. Still, this approach has had some notable successes recently. For example, a novel 27 amino acid sequence αββ motif with a predefined backbone was designed (Dahiyat and Mayo 1997, Science 278: 82-87), a novel iron superoxide dismutase was designed (Pinto et al. 1997, Proc. Natl. Acad. Sci. USA 94: 5562-5567), a novel 93 amino acid protein fold not found in nature, “Top7” was designed (Kuhlman et al. 2003, Science 302: 1364-1368), addition of enzymatic activity (triose phosphate isomerase) into a nonenzyme scaffold (ribose binding protein) was achieved through protein design (Dwyer et al. 2003, Science 304: 1967-1971), novel sensor proteins were designed (Looger et al. 2003, Nature 423: 185-190), and a therapeutic protein variant (dominant negative TNF-alpha variant) has been designed (Steed et al 2003, Science 301: 1895-1898).

The field is becoming increasingly aware that the empirical models used to score each design may not be sufficiently good to separate the best 10 or 20 designs from the others. This was highlighted in a recent paper pointing out how some models are used to make predictions far from their optimal regimes (Jaramillo and Wodak 2005, Biophys. J 88: 156-171). Practitioners have a desire to synthesize and test more than ˜10 of their in silico designs, perhaps 100 to 1000 or even 10000 proteins instead, to avoid missing possible solutions to the design problem due to only a slight error in the model.

We have now discovered a novel method to synthesize a large number of DNA sequences at low cost, which will enable protein designers to build, at reasonable cost and in a reasonable time, a far greater portion or all of their high scoring designs, perhaps 10⁴ specific sequences or more. This has the potential to yield solutions in situations where model accuracy is not perfect, and a “good answer” is in fact somewhere in the rank-ordered list between what could previously be tested (˜10 designs) and what we will enable to be tested (˜10,000 or more designs).

Thus, in silico designs can be made to produce a library of constructs that can serve as a pool or plural separate species that can be tested or selected for a good candidate, or can serve as a starting places for other purposeful design iterations or for evolutionary techniques utilizing random mutagenesis. A screen or selection can be applied to the pool, and if necessary, the process (starting from design or another library expansion) can be iterated. This general strategy is referred to herein as “rational diversity” and emphasizes the importance of a mechanistic model (“rational”) in the initial library design.

Design is a necessity for what can't be done (or can't be done in a reasonable amount of time) by mutation or evolution. Fundamentally, this arises from the difference in library sizes for computational versus experimental screens. Natural biological evolution and derivative laboratory techniques like directed evolution have two important constraints. First, intermediates must be viable (or functional). Nonviability (nonfunctionality) breaks the chain. Second, evolutionary time is not sufficient to search sequence space exhaustively. However, synthetic protein design does not evolve in the Darwinian sense and therefore doesn't have to descend from another successful design, and this greatly expands the possibilities for protein design.

RosettaDesign from the Baker group at the University of Washington is a model case for how protein design software works. One begins with some understanding of how the backbone conformation of a protein relates to whatever function is being designed or engineered (for example how it forms or doesn't form a properly folded structure, binding pocket, catalytic site, etc.). The program takes the spatial position of a desired protein backbone as input. It then searches all possible amino acid sequences to find those that have the minimum energy for the given backbone conformation. The energy model is a combination of semiempirical (Lennard-Jones) and fully empirical (implicit solvation) models. The current version of RosettaDesign not only can search all possible sequences, but determines whether or not each sequence will be stable in the target conformation, discarding those sequences that are not (Kuhlman et al. 2003, Science 302: 1364-1368).

Generally, the invention provides polynucleotide, protein, and library production techniques that may be used in various fields and contexts to produce useful biological constructs. Exemplary uses for protein design include, for example, design of proteins having novel characteristics including biochemical and/or biophysical properties. Another example is for the design of novel catalytic RNAs. In one embodiment, the methods described herein may be used to develop improved human therapeutics, for example, by designing backbones around active site residues and mutating residues in silico to produce variants with desired characteristics such as higher binding affinity, improved stability, lower immunogenicity, better bioavailability, or ease of manufacture while maintaining functionality. In another embodiment, the methods described herein may be used to develop novel industrial enzymes, for example, by designing active sites to carry out desired chemical transformation, and then designing a backbone scaffold to hold the novel active site in an active conformation. Exemplary applications for industrial enzymes include chemical synthesis, pulp and paper bleaching, conversion of biomass to energy, etc. In another embodiment, the methods disclosed herein may be used to develop bi-functional or multifunctional proteins. For example, multivalent, high-affinity binders, may be developed by designing linkers to optimally connect binding domains yielding a construct with, e.g., the highest possible affinity, or a slow off rate. Additionally, the methods described herein may be used to develop combinations of a binding domain, linker and catalytic domain that result in optimal catalytic efficiency. In yet another embodiment, the methods described herein may be used to develop “minimal proteins.” For example, the backbone of the functional area(s) of a protein may be fixed and the chains of this region may be connected with the smallest possible backbone that results in a single, stable molecule. The sequence of the polypeptide may be further optimized to maintain the structure of the backbone. Such minimal proteins may facilitate protein manufacturing and yield proteins with greater stability or higher rates of diffusion.

In an exemplary embodiment, large numbers of protein design variants may be expressed and subjected to a screen, or preferably a selection process, to identify variants exhibiting a desired characteristic. In various embodiments, at least 10, 100, 1,000, 10,000, 100,000 or more variants may be screened for a desired characteristic. Such variants may optionally be selected based on an in silico prescreen that produces a rank ordered list of variants obtained from analysis of a large library of possible variants.

3. Generation of Libraries of Variants

By computationally screening very large libraries of mutants (variants), greater diversity of protein sequences can be screened (i.e. a larger sampling of sequence space), leading to greater improvements in protein function. Further, fewer mutants may need to be tested experimentally to screen a given library size, reducing the cost and difficulty of protein engineering. By using computational methods to pre-screen a protein library, the computational features of speed and efficiency are combined with the ability of experimental library screening to create new activities in proteins for which appropriate computational models and structure-function relationships are unclear.

In addition, as is more fully outlined below, the libraries may be biased in any number of ways, allowing the generation of libraries that vary in their focus; for example, domains, individual residues, surface residues, subsets of residues, active or binding sites, etc., may all be varied or kept constant as desired.

Accordingly, the present invention provides methods for generating secondary libraries of scaffold protein variants. Protein as used herein is meant to encompass at least two amino acids linked together by a peptide bond, including, polypeptides, oligopeptides, peptides and variously derivatized polypeptides such as phosphorylated or glycosylated proteins. The peptidyl group may comprise naturally occurring amino acids and peptide bonds, or synthetic peptidomimetic structures, i.e. “analogs”, such as peptoids (see Simon et al., PNAS USA 89(20):9367 (1992)). The amino acids may either be naturally occurring or non-naturally occurring; as will be appreciated by those in the art, any structure for which a set of rotamers is known or can be generated can be used as an amino acid. The side chains may be in either the (R) or the (S) configuration. In a preferred embodiment, the amino acids are in the (S) or L-configuration.

The scaffold protein may be any protein, but preferred proteins are those for which a three dimensional structure is known or can be generated; that is, for which there are three dimensional coordinates for each atom of the protein. Generally this can be determined using X-ray crystallographic techniques, NMR techniques, de novo modelling, homology modelling, etc. In general, if X-ray structures are used, structures at 2 Å resolution or better are preferred, but not required.

The scaffold proteins may be from any organism, including prokaryotes and eukaryotes, with enzymes from bacteria, fungi, extremeophiles such as the archebacteria, insects, fish, animals (particularly mammals and particularly human) and birds all possible.

Thus, by “scaffold protein” herein is meant a protein for which a library of variants is desired. As will be appreciated by those in the art, any number of scaffold proteins find use in the present invention. Specifically included within the definition of “protein” are fragments and domains of known proteins, including functional domains such as enzymatic domains, binding domains, etc., and smaller fragments, such as turns, loops, etc. That is, portions of proteins may be used as well. In addition, “protein” as used herein includes proteins, oligopeptides and peptides. In addition, protein variants, i.e. non-naturally occurring protein analog structures, may be used. Suitable proteins include, but are not limited to, industrial and pharmaceutical proteins, including ligands, cell surface receptors, antigens, antibodies, cytokines, hormones, transcription factors, signaling modules, cytoskeletal proteins and enzymes. Suitable classes of enzymes include, but are not limited to, hydrolases such as proteases, carbohydrases, lipases; isomerases such as racemases, epimerases, tautomerases, or mutases; transferases, kinases, oxidoreductases, and phophatases. Suitable enzymes are listed in the Swiss-Prot enzyme database. Suitable protein backbones include, but are not limited to, all of those found in the protein data base compiled and serviced by the Research Collaboratory for Structural Bioinformatics (RCSB, formerly the Brookhaven National Lab).

Specifically, preferred scaffold proteins include, but are not limited to, those with known structures (including variants) including cytokines (IL-1ra (+receptor complex), IL-1 (receptor alone), IL-1a, IL-1b (including variants and or receptor complex), IL-2, IL-3, IL-4, IL-5, IL-6, IL-8, IL-10, IFN-β, INF-γ, IFN-α-2a; IFN-α-2B, TNF-α; CD40 ligand (chk), Human Obesity Protein Leptin, Granulocyte Colony-Stimulating Factor, Bone Morphogenetic Protein-7, Ciliary Neurotrophic Factor, Granulocyte-Macrophage Colony-Stimulating Factor, Monocyte Chemoattractant Protein 1, Macrophage Migration Inhibitory Factor, Human Glycosylation-Inhibiting Factor, Human Rantes, Human Macrophage Inflammatory Protein 1 Beta, human growth hormone, Leukemia Inhibitory Factor, Human Melanoma Growth Stimulatory Activity, neutrophil activating peptide-2, Cc-Chemokine Mcp-3, Platelet Factor M2, Neutrophil Activating Peptide 2, Eotaxin, Stromal Cell-Derived Factor-1, Insulin, Insulin-like Growth Factor I, Insulin-like Growth Factor II, Transforming Growth Factor B1, Transforming Growth Factor B2, Transforming Growth Factor B3, Transforming Growth Factor A, Vascular Endothelial growth factor (VEGF), acidic Fibroblast growth factor, basic Fibroblast growth factor, Endothelial growth factor, Nerve growth factor, Brain Derived Neurotrophic Factor, Ciliary Neurotrophic Factor, Platelet Derived Growth Factor, Human Hepatocyte Growth Factor, Glial Cell-Derived Neurotrophic Factor, (as well as the at least 55 cytokines in PDB)); Erythropoietin; other extracellular signalling moeities, including, but not limited to, hedgehog Sonic, hedgehog Desert, hedgehog Indian, hCG; coaguation factors including, but not limited to, TPA and Factor VIIa; transcription factors, including but not limited to, p53, p53 tetramerization domain, Zn fingers (of which more than 12 have structures), homeodomains (of which 8 have structures), leucine zippers (of which 4 have structures); antibodies, including, but not limited to, cFv; viral proteins, including, but not limited to, hemagglutinin trimerization domain and hiv Gp41 ectodomain (fusion domain); intracellular signalling modules, including, but not limited to, SH2 domains (of which 8 structures are known), SH3 domains (of which 11 have structures), and Pleckstin Homology Domains; receptors, including, but not limited to, the extracellular Region Of Human Tissue Factor Cytokine-Binding Region Of Gp130, G-CSF receptor, erythropoietin receptor, Fibroblast Growth Factor receptor, TNF receptor, IL-1 receptor, IL-1 receptor/IL1ra complex, IL-4 receptor, INF-.gamma. receptor alpha chain, MHC Class I, MHC Class II, T Cell Receptor, Insulin receptor, insulin receptor tyrosine kinase and human growth hormone receptor.

Once a scaffold protein is chosen, a library may be generated, typically using known or to be developed computational processing techniques. Generally speaking, in some embodiments, the goal of the computational processing is to determine a set of optimized protein sequences. By “optimized protein sequence” herein is meant a sequence that best fits the mathematical equations of the computational process. As will be appreciated by those in the art, a global optimized sequence is the one sequence that best fits the equations (for example, when protein design automation (PDA) is used, the global optimized sequence is the sequence that best fits Equation 1, below); i.e. the sequence that has the lowest energy of any possible sequence. However, there are any number of sequences that are not the global minimum but that have low energies.

The libraries can be generated in a variety of ways. In essence, any methods that can result in either the relative ranking of the possible sequences of a protein based on measurable stability parameters, or a list of suitable sequences can be used. As will be appreciated by those in the art, any of the methods described herein or known in the art may be used alone, or in combination with other methods.

Generally, there are a variety of computational methods that can be used to generate a library. In a preferred embodiment, sequence based methods are used. Alternatively, structure based methods, such as protein design automation (PDA), described in detail below, are used.

In a preferred embodiment, the scaffold protein is an enzyme and highly accurate electrostatic models can be used for enzyme active site residue scoring to improve enzyme active site libraries (see Warshel, Computer Modeling of Chemical Reactions in Enzymes and Solutions, Wiley & Sons, New York, (1991), hereby expressly incorporated by reference). These accurate models can assess the relative energies of sequences with high precision, but are computationally intensive.

Similarly, molecular dynamics calculations can be used to computationally screen sequences by individually calculating mutant sequence scores and compiling a rank ordered list.

In a preferred embodiment, residue pair potentials can be used to score sequences (Miyazawa et al., Macromolecules 18(3):534-552 (1985), expressly incorporated by reference) during computational screening.

In a preferred embodiment, sequence profile scores (Bowie et al., Science 253(5016):164-70 (1991), incorporated by reference) and/or potentials of mean force (Hendlich et al., J. Mol. Biol. 216(1):167-180 (1990), also incorporated by reference) can also be calculated to score sequences. These methods assess the match between a sequence and a 3D protein structure and hence can act to screen for fidelity to the protein structure. By using different scoring functions to rank sequences, different regions of sequence space can be sampled in the computational screen.

Furthermore, scoring functions can be used to screen for sequences that would create metal or co-factor binding sites in the protein (Hellinga, Fold Des. 3(1):R1-8 (1998), hereby expressly incorporated by reference). Similarly, scoring functions can be used to screen for sequences that would create disulfide bonds in the protein. These potentials attempt to specifically modify a protein structure to introduce a new structural motif.

In a preferred embodiment, sequence and/or structural alignment programs can be used to generate libraries. As is known in the art, there are a number of sequence-based alignment programs; including for example, Smith-Waterman searches, Needleman-Wunsch, Double Affine Smith-Waterman, frame search, Gribskov/GCG profile search, Gribskov/GCG profile scan, profile frame search, Bucher generalized profiles, Hidden Markov models, Hframe, Double Frame, Blast, Psi-Blast, Clustal, and GeneWise.

The source of the sequences can vary widely, and include taking sequences from one or more of the known databases, including, but not limited to, SCOP (Hubbard, et al., Nucleic Acids Res 27(1):254-256. (1999)); PFAM (Bateman, et al., Nucleic Acids Res 27(1):260-262. (1999)); VAST (Gibrat, et al., Curr Opin Struct Biol 6(3):377-385. (1996)); CATH (Orengo, et al., Structure 5(8):1093-1108. (1997)); PhD Predictor (world wide web at embl-heidelberg.de/predictprotein/predictprotein.html); Prosite (Hofmann, et al., Nucleic Acids Res 27(1):215-219. (1999)); PIR (world wide web at mips.biochem.mpg.de/proj/protseqdb/); GenBank (world wide web at ncbi.nlm.nih.gov/); PDB (world wide web at rcsb.org) and BIND (Bader, et al., Nucleic Acids Res 29(1):242-245 (2001)).

In addition, sequences from these databases can be subjected to contiguous analysis or gene prediction; see Wheeler, et al., Nucleic Acids Res 28(1):10-14. (2000) and Burge and Karlin, J Mol Biol 268(1):78-94. (1997).

As is known in the art, there are a number of sequence alignment methodologies that can be used. For example, sequence homology based alignment methods can be used to create sequence alignments of proteins related to the target structure (Altschul et al., J. Mol. Biol. 215(3):403 (1990), incorporated by reference). These sequence alignments are then examined to determine the observed sequence variations. These sequence variations are tabulated to define a primary library. In addition, as is further outlined below, these methods can also be used to generate secondary libraries.

Sequence based alignments can be used in a variety of ways. For example, a number of related proteins can be aligned, as is known in the art, and the “variable” and “conserved” residues defined; that is, the residues that vary or remain identical between the family members can be defined. These results can be used to generate a probability table. Alternatively, the allowed sequence variations can be used to define the amino acids considered at each position during the computational screening. Another variation is to bias the score for amino acids that occur in the sequence alignment, thereby increasing the likelihood that they are found during computational screening but still allowing consideration of other amino acids. This bias would result in a focused primary library but would not eliminate from consideration amino acids not found in the alignment. In addition, a number of other types of bias may be introduced. For example, diversity may be forced; that is, a “conserved” residue is chosen and altered to force diversity on the protein and thus sample a greater portion of the sequence space. Alternatively, the positions of high variability between family members (i.e. low conservation) can be randomized, either using all or a subset of amino acids. Similarly, outlier residues, either positional outliers or side chain outliers, may be eliminated.

Similarly, structural alignment of structurally related proteins can be done to generate sequence alignments. There are a wide variety of such structural alignment programs known. See for example VAST from the NCBI (world wide web at ncbi.nlm.nih.gov:80/StructureNAST/vast.shtml); SSAP (Orengo and Taylor, Methods Enzymol 266(617-635 (1996)) SARF2 (Alexandrov, Protein Eng 9(9):727-732. (1996)) CE (Shindyalov and Bourne, Protein Eng 11(9):739-747. (1998)); (Orengo et al., Structure 5(8):1093-108 (1997); Dali (Holm et al., Nucleic Acid Res. 26(1):316-9 (1998), all of which are incorporated by reference). These structurally-generated sequence alignments can then be examined to determine the observed sequence variations.

In certain embodiments, libraries can be generated by predicting secondary structure from sequence, and then selecting sequences that are compatible with the predicted secondary structure. There are a number of secondary structure prediction methods, including, but not limited to, threading (Bryant and Altschul, Curr Opin Struct Biol 5(2):236-244. (1995)), Profile 3D (Bowie, et al., Methods Enzymol 266(598-616 (1996); MONSSTER (Skolnick, et al., J Mol Biol 265(2):217-241. (1997); Rosetta (Simons, et al., Proteins 37(S3):171-176 (1999); PSI-BLAST (Altschul and Koonin, Trends Biochem Sci 23(11):444-447. (1998)); Impala (Schaffer, et al., Bioinformatics 15(12):1000-1011. (1999)); HMMER (McClure, et al., Proc Int Conf Intell Syst Mol Biol 4(155-164 (1996)); Clustal W (world wide web at ebi.ac.uk/clustalw/); BLAST (Altschul, et al., J Mol Biol 215(3):403-410. (1990)), helix-coil transition theory (Munoz and Serrano, Biopolymers 41:495, 1997), neural networks, local structure alignment and others (e.g., see in Selbig et al., Bioinformatics 15:1039, 1999).

Similarly, as outlined above, other computational methods are known, including, but not limited to, sequence profiling (Bowie and Eisenberg, Science 253(5016): 164-70, (1991)), rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)); and residue pair potentials (Jones, Protein Science 3: 567-574, (1994); PROSA (Heindlich et al., J. Mol. Biol. 216:167-180 (1990); THREADER (Jones et al., Nature 358:86-89 (1992), and other inverse folding methods such as those described by Simons et al. (Proteins, 34:535-543, 1999), Levitt and Gerstein (PNAS USA, 95:5913-5920, 1998), Godzik et al., PNAS, V89, PP 12098-102; Godzik and Skolnick (PNAS USA, 89:12098-102, 1992), Godzik et al. (J. Mol. Biol. 227:227-38, 1992) and two profile methods (Gribskov et al. PNAS 84:4355-4358 (1987) and Fischer and Eisenberg, Protein Sci. 5:947-955 (1996), Rice and Eisenberg J. Mol. Biol. 267:1026-1038(1997)), all of which are expressly incorporated by reference. In addition, other computational methods such as those described by Koehl and Levitt (J. Mol. Biol. 293:1161-1181 (1999); J. Mol. Biol. 293:1183-1193 (1999); expressly incorporated by reference) can be used to create a protein sequence library for improved properties and function.

In addition, there are computational methods based on forcefield calculations such as SCMF that can be used as well for SCMF, see Delarue et la. Pac. Symp. Biocomput. 109-21 (1997), Koehl et al., J. Mol. Biol. 239:249 (1994); Koehl et al., Nat. Struc. Biol. 2:163 (1995); Koehl et al., Curr. Opin. Struct. Biol. 6:222 (1996); Koehl et al., J. Mol. Bio. 293:1183 (1999); Koehl et al., J. Mol. Biol. 293:1161 (1999); Lee J. Mol. Biol. 236:918 (1994); and Vasquez Biopolymers 36:53-70 (1995); all of which are expressly incorporated by reference. Other forcefield calculations that can be used to optimize the conformation of a sequence within a computational method, or to generate de novo optimized sequences as outlined herein include, but are not limited to, OPLS-AA (Jorgensen, et al., J. Am. Chem. Soc. (1996), v 118, pp 11225-11236; Jorgensen, W. L.; BOSS, Version 4.1; Yale University: New Haven, Conn. (1999)); OPLS (Jorgensen, et al., J. Am. Chem. Soc. (1988), v 110, pp 1657ff; Jorgensen, et al., J. Am. Chem. Soc. (1990), v 112, pp 4768ff); UNRES (United Residue Forcefield; Liwo, et al., Protein Science (1993), v 2, pp1697-1714; Liwo, et al., Protein Science (1993), v 2, pp1715-1731; Liwo, et al., J. Comp. Chem. (1997), v 18, pp849-873; Liwo, et al., J. Comp. Chem. (1997), v 18, pp874-884; Liwo, et al., J. Comp. Chem. (1998), v 19, pp259-276; Forcefield for Protein Structure Prediction (Liwo, et al., Proc. Natl. Acad. Sci. USA (1999), v 96, pp5482-5485); ECEPP/3 (Liwo et al., J Protein Chem 1994 May;13(4):375-80); AMBER 1.1 force field (Weiner, et al., J. Am. Chem. Soc. v106, pp765-784); AMBER 3.0 force field (U. C. Singh et al., Proc. Natl. Acad. Sci. USA. 82:755-759); CHARMM and CHARMM22 (Brooks, et al., J. Comp. Chem. v4, pp 187-217); cvff.0 (Dauber-Osguthorpe, et al., (1988) Proteins: Structure, Function and Genetics, v4,pp31-47); cff91 (Maple, et al., J. Comp. Chem. v15, 162-182); also, the DISCOVER (cvff and cff91) and AMBER forcefields are used in the INSIGHT molecular modeling package (Biosym/MSI, San Diego Calif.) and HARMM is used in the QUANTA molecular modeling package (Biosym/MSI, San Diego Calif.), all of which are expressly incorporated by reference.

In a preferred embodiment, the computational method used to generate the primary library is Protein Design Automation (PDA), as is described in U.S. Pat. No. 6,269,312 and PCT Publication No. WO 98/47089, both of which are expressly incorporated herein by reference. Briefly, PDA can be described as follows. A known protein structure is used as the starting point. The residues to be optimized are then identified, which may be the entire sequence or subset(s) thereof. The side chains of any positions to be varied are then removed. The resulting structure consisting of the protein backbone and the remaining sidechains is called the template. Each variable residue position is then preferably classified as a core residue, a surface residue, or a boundary residue; each classification defines a subset of possible amino acid residues for the position (for example, core residues generally will be selected from the set of hydrophobic residues, surface residues generally will be selected from the hydrophilic residues, and boundary residues may be either). Each amino acid can be represented by a discrete set of all allowed conformers of each side chain, called rotamers. Thus, to arrive at an optimal sequence for a backbone, all possible sequences of rotamers must be screened, where each backbone position can be occupied either by each amino acid in all its possible rotameric states, or a subset of amino acids, and thus a subset of rotamers.

Two sets of interactions are then calculated for each rotamer at every position: the interaction of the rotamer side chain with all or part of the backbone (the “singles” energy, also called the rotamer/template or rotamer/backbone energy), and the interaction of the rotamer side chain with all other possible rotamers at every other position or a subset of the other positions (the “doubles” energy, also called the rotamer/rotamer energy). The energy of each of these interactions is calculated through the use of a variety of scoring functions, which include the energy of van der Waal's forces, the energy of hydrogen bonding, the energy of secondary structure propensity, the energy of surface area solvation and the electrostatics. Thus, the total energy of each rotamer interaction, both with the backbone and other rotamers, is calculated, and stored in a matrix form.

The discrete nature of rotamer sets allows a simple calculation of the number of rotamer sequences to be tested. A backbone of length n with m possible rotamers per position will have mⁿ possible rotamer sequences, a number which grows exponentially with sequence length and renders the calculations either unwieldy or impossible in real time. Accordingly, to solve this combinatorial search problem, a “Dead End Elimination” (DEE) calculation is performed. The DEE calculation is based on the fact that if the worst total interaction of a first rotamer is still better than the best total interaction of a second rotamer, then the second rotamer cannot be part of the global optimum solution. Since the energies of all rotamers have already been calculated, the DEE approach only requires sums over the sequence length to test and eliminate rotamers, which speeds up the calculations considerably. DEE can be rerun comparing pairs of rotamers, or combinations of rotamers, which will eventually result in the determination of a single sequence which represents the global optimum energy.

Once the global solution has been found, a Monte Carlo search may be done to generate a rank-ordered list of sequences in the neighborhood of the DEE solution. Starting at the DEE solution, random positions are changed to other rotamers, and the new sequence energy is calculated. If the new sequence meets the criteria for acceptance, it is used as a starting point for another jump. After a predetermined number of jumps, a rank-ordered list of sequences is generated. Monte Carlo searching is a sampling technique to explore sequence space around the global minimum or to find new local minima distant in sequence space. As is more additionally outlined below, there are other sampling techniques that can be used, including Boltzman sampling, genetic algorithm techniques and simulated annealing. In addition, for all the sampling techniques, the kinds of jumps allowed can be altered (e.g. random jumps to random residues, biased jumps (to or away from wild-type, for example), jumps to biased residues (to or away from similar residues, for example), etc.). Similarly, for all the sampling techniques, the acceptance criteria of whether a sampling jump is accepted can be altered.

As outlined in U.S. Pat. No. 6,269,312, the protein backbone (comprising (for a naturally occuring protein) the nitrogen, the carbonyl carbon, the α-carbon, and the carbonyl oxygen, along with the direction of the vector from the α-carbon to the β-carbon) may be altered prior to the computational analysis, by varying a set of parameters called supersecondary structure parameters.

Once a protein structure backbone is generated (with alterations, as outlined above) and input into the computer, explicit hydrogens are added if not included within the structure (for example, if the structure was generated by X-ray crystallography, hydrogens must be added). After hydrogen addition, energy minimization of the structure is run, to relax the hydrogens as well as the other atoms, bond angles and bond lengths. In a preferred embodiment, this is done by doing a number of steps of conjugate gradient minimization (Mayo et al., J. Phys. Chem. 94:8897 (1990)) of atomic coordinate positions to minimize the Dreiding force field with no electrostatics. Generally from about 10 to about 250 steps is preferred, with about 50 being most preferred.

The protein backbone structure contains at least one variable residue position. As is known in the art, the residues, or amino acids, of proteins are generally sequentially numbered starting with the N-terminus of the protein. Thus a protein having a methionine at it's N-terminus is said to have a methionine at residue or amino acid position 1, with the next residues as 2, 3, 4, etc. At each position, the wild type (i.e. naturally occuring) protein may have one of at least 20 amino acids, in any number of rotamers. By “variable residue position” herein is meant an amino acid position of the protein to be designed that is not fixed in the design method as a specific residue or rotamer, generally the wild-type residue or rotamer.

In a preferred embodiment, all of the residue positions of the protein are variable. That is, every amino acid side chain may be altered in the methods of the present invention. This is particularly desirable for smaller proteins, although the present methods allow the design of larger proteins as well. While there is no theoretical limit to the length of the protein which may be designed this way, there is a practical computational limit.

In an alternate preferred embodiment, only some of the residue positions of the protein are variable, and the remainder are “fixed”, that is, they are identified in the three dimensional structure as being in a set conformation. In some embodiments, a fixed position is left in its original conformation (which may or may not correlate to a specific rotamer of the rotamer library being used). Alternatively, residues may be fixed as a non-wild type residue; for example, when known site-directed mutagenesis techniques have shown that a particular residue is desirable (for example, to eliminate a proteolytic site or alter the substrate specificity of an enzyme), the residue may be fixed as a particular amino acid. Alternatively, the methods of the present invention may be used to evaluate mutations de novo, as is discussed below. In an alternate preferred embodiment, a fixed position may be “floated”; the amino acid at that position is fixed, but different rotamers of that amino acid are tested. In this embodiment, the variable residues may be at least one, or anywhere from 0.1% to 99.9% of the total number of residues. Thus, for example, it may be possible to change only a few (or one) residues, or most of the residues, with all possibilities in between.

In a preferred embodiment, residues which can be fixed include, but are not limited to, structurally or biologically functional residues; alternatively, biologically functional residues may specifically not be fixed. For example, residues which are known to be important for biological activity, such as the residues which form the active site of an enzyme, the substrate binding site of an enzyme, the binding site for a binding partner (ligand/receptor, antigen/antibody, etc.), phosphorylation or glycosylation sites which are crucial to biological function, or structurally important residues, such as disulfide bridges, metal binding sites, critical hydrogen bonding residues, residues critical for backbone conformation such as proline or glycine, residues critical for packing interactions, etc. may all be fixed in a conformation or as a single rotamer, or “floated”.

Similarly, residues which may be chosen as variable residues may be those that confer undesirable biological attributes, such as susceptibility to proteolytic degradation, dimerization or aggregation sites, glycosylation sites which may lead to immune responses, unwanted binding activity, unwanted allostery, undesirable enzyme activity but with a preservation of binding, etc.

In a preferred embodiment, each variable position is classified as either a core, surface or boundary residue position, although in some cases, as explained below, the variable position may be set to glycine to minimize backbone strain. In addition, as outlined herein, residues need not be classified, they can be chosen as variable and any set of amino acids may be used. Any combination of core, surface and boundary positions can be utilized: core, surface and boundary residues; core and surface residues; core and boundary residues, and surface and boundary residues, as well as core residues alone, surface residues alone, or boundary residues alone.

The classification of residue positions as core, surface or boundary may be done in several ways, as will be appreciated by those in the art. In a preferred embodiment, the classification is done via a visual scan of the original protein backbone structure, including the side chains, and assigning a classification based on a subjective evaluation of one skilled in the art of protein modelling. Alternatively, a preferred embodiment utilizes an assessment of the orientation of the Cα-Cβ vectors relative to a solvent accessible surface computed using only the template Cα atoms, as outlined in U.S. Pat. No. No. 6,269,312 and PCT Publication No. WO 98/47089. Alternatively, a surface area calculation can be done.

Once each variable position is classified as either core, surface or boundary, a set of amino acid side chains, and thus a set of rotamers, is assigned to each position. That is, the set of possible amino acid side chains that the program will allow to be considered at any particular position is chosen. Subsequently, once the possible amino acid side chains are chosen, the set of rotamers that will be evaluated at a particular position can be determined. Thus, a core residue will generally be selected from the group of hydrophobic residues consisting of alanine, valine, isoleucine, leucine, phenylalanine, tyrosine, tryptophan, and methionine (in some embodiments, when the a scaling factor of the van der Waals scoring function, described below, is low, methionine is removed from the set), and the rotamer set for each core position potentially includes rotamers for these eight amino acid side chains (all the rotamers if a backbone independent library is used, and subsets if a rotamer dependent backbone is used). Similarly, surface positions are generally selected from the group of hydrophilic residues consisting of alanine, serine, threonine, aspartic acid, asparagine, glutamine, glutamic acid, arginine, lysine and histidine. The rotamer set for each surface position thus includes rotamers for these ten residues. Finally, boundary positions are generally chosen from alanine, serine, threonine, aspartic acid, asparagine, glutamine, glutamic acid, arginine, lysine histidine, valine, isoleucine, leucine, phenylalanine, tyrosine, tryptophan, and methionine. The rotamer set for each boundary position thus potentially includes every rotamer for these seventeen residues (assuming cysteine, glycine and proline are not used, although they can be). Additionally, in some preferred embodiments, a set of 18 naturally occuring amino acids (all except cysteine and proline, which are known to be particularly disruptive) are used.

Thus, as will be appreciated by those in the art, there is a computational benefit to classifying the residue positions, as it decreases the number of calculations. It should also be noted that there may be situations where the sets of core, boundary and surface residues are altered from those described above; for example, under some circumstances, one or more amino acids is either added or subtracted from the set of allowed amino acids. For example, some proteins which dimerize or multimerize, or have ligand binding sites, may contain hydrophobic surface residues, etc. In addition, residues that do not allow helix “capping” or the favorable interaction with an α-helix dipole may be subtracted from a set of allowed residues. This modification of amino acid groups is done on a residue by residue basis.

In a preferred embodiment, proline, cysteine and glycine are not included in the list of possible amino acid side chains, and thus the rotamers for these side chains are not used. However, in a preferred embodiment, when the variable residue position has a φ angle (that is, the dihedral angle defined by 1) the carbonyl carbon of the preceding amino acid; 2) the nitrogen atom of the current residue; 3) the α-carbon of the current residue; and 4) the carbonyl carbon of the current residue) greater than 0°, the position is set to glycine to minimize backbone strain.

Once the group of potential rotamers is assigned for each variable residue position, processing proceeds as outlined in U.S. Pat. No. 6,269,312 and PCT Publication No. WO 98/47089. This processing step entails analyzing interactions of the rotamers with each other and with the protein backbone to generate optimized protein sequences. Simplistically, the processing initially comprises the use of a number of scoring functions to calculate energies of interactions of the rotamers, either to the backbone itself or other rotamers. Preferred PDA scoring functions include, but are not limited to, a Van der Waals potential scoring function, a hydrogen bond potential scoring function, an atomic solvation scoring function, a secondary structure propensity scoring function and an electrostatic scoring function. As is further described below, at least one scoring function is used to score each position, although the scoring functions may differ depending on the position classification or other considerations, like favorable interaction with an α-helix dipole. As outlined below, the total energy which is used in the calculations is the sum of the energy of each scoring function used at a particular position, as is generally shown in Equation 1:
Eₜₒₜₐₗ=nE_{vdw}+nEₐₛ+nEₕ−bonding+nEₛₛ+nE_{elec} Equation 1

In Equation 1, the total energy is the sum of the energy of the van der Waals potential (E_{vdw}), the energy of atomic solvation (Eₐₛ), the energy of hydrogen bonding (Eₕ-bonding), the energy of secondary structure (Eₛₛ) and the energy of electrostatic interaction (E_{elec}). The term n is either 0 or 1, depending on whether the term is to be considered for the particular residue position.

As outlined in U.S. Pat. No. 6,269,312 and PCT Publication No. WO 98/47089, any combination of these scoring functions, either alone or in combination, may be used. Once the scoring functions to be used are identified for each variable position, the preferred first step in the computational analysis comprises the determination of the interaction of each possible rotamer with all or part of the remainder of the protein. That is, the energy of interaction, as measured by one or more of the scoring functions, of each possible rotamer at each variable residue position with either the backbone or other rotamers, is calculated. In a preferred embodiment, the interaction of each rotamer with the entire remainder of the protein, i.e. both the entire template and all other rotamers, is done. However, as outlined above, it is possible to only model a portion of a protein, for example a domain of a larger protein, and thus in some cases, not all of the protein need be considered. The term “portion”, as used herein, with regard to a protein refers to a fragment of that protein. This fragment may range in size from 10 amino acid residues to the entire amino acid sequence minus one amino acid. Accordingly, the term “portion”, as used herein, with regard to a nucleic refers to a fragment of that nucleic acid. This fragment may range in size from 10 nucleotides to the entire nucleic acid sequence minus one nucleotide.

In a preferred embodiment, the first step of the computational processing is done by calculating two sets of interactions for each rotamer at every position: the interaction of the rotamer side chain with the template or backbone (the “singles” energy), and the interaction of the rotamer side chain with all other possible rotamers at every other position (the “doubles” energy), whether that position is varied or floated. It should be understood that the backbone in this case includes both the atoms of the protein structure backbone, as well as the atoms of any fixed residues, wherein the fixed residues are defined as a particular conformation of an amino acid.

Thus, “singles” (rotamer/template) energies are calculated for the interaction of every possible rotamer at every variable residue position with the backbone, using some or all of the scoring functions. Thus, for the hydrogen bonding scoring function, every hydrogen bonding atom of the rotamer and every hydrogen bonding atom of the backbone is evaluated, and the EHB is calculated for each possible rotamer at every variable position. Similarly, for the van der Waals scoring function, every atom of the rotamer is compared to every atom of the template (generally excluding the backbone atoms of its own residue), and the E_{vdW} is calculated for each possible rotamer at every variable residue position. In addition, generally no van der Waals energy is calculated if the atoms are connected by three bonds or less. For the atomic salvation scoring function, the surface of the rotamer is measured against the surface of the template, and the Eₐₛ for each possible rotamer at every variable residue position is calculated. The secondary structure propensity scoring function is also considered as a singles energy, and thus the total singles energy may contain an Eₛₛ term. As will be appreciated by those in the art, many of these energy terms will be close to zero, depending on the physical distance between the rotamer and the template position; that is, the farther apart the two moieties, the lower the energy.

For the calculation of “doubles” energy (rotamer/rotamer), the interaction energy of each possible rotamer is compared with every possible rotamer at all other variable residue positions. Thus, “doubles” energies are calculated for the interaction of every possible rotamer at every variable residue position with every possible rotamer at every other variable residue position, using some or all of the scoring functions. Thus, for the hydrogen bonding scoring function, every hydrogen bonding atom of the first rotamer and every hydrogen bonding atom of every possible second rotamer is evaluated, and the E_{HB} is calculated for each possible rotamer pair for any two variable positions. Similarly, for the van der Waals scoring function, every atom of the first rotamer is compared to every atom of every possible second rotamer, and the E_{vdw} is calculated for each possible rotamer pair at every two variable residue positions. For the atomic solvation scoring function, the surface of the first rotamer is measured against the surface of every possible second rotamer, and the Eₐₛ for each possible rotamer pair at every two variable residue positions is calculated. The secondary structure propensity scoring function need not be run as a “doubles” energy, as it is considered as a component of the “singles” energy. As will be appreciated by those in the art, many of these double energy terms will be close to zero, depending on the physical distance between the first rotamer and the second rotamer; that is, the farther apart the two moieties, the lower the energy.

In addition, as will be appreciated by those in the art, a variety of force fields that can be used in the PCA calculations can be used, including, but not limited to, Dreiding I and Dreiding II (Mayo et al, J. Phys. Chem. 948897 (1990)), AMBER (Weiner et al., J. Amer. Chem. Soc. 106:765 (1984) and Weiner et al., J. Comp. Chem. 106:230 (1986)), MM2 (Allinger J. Chem. Soc. 99:8127 (1977), Liljefors et al., J. Corn. Chem. 8:1051 (1987)); MMP2 (Sprague et al., J. Comp. Chem. 8:581 (1987)); CHARMM (Brooks et al., J. Comp. Chem. 106:187 (1983)); GROMOS; and MM3 (Allinger et al., J. Amer. Chem. Soc. 111:8551 (1989)), OPLS-M (Jorgensen, et al., J. Am. Chem. Soc. (1996), v 118, pp 11225-11236; Jorgensen, W. L.; BOSS, Version 4.1; Yale University: New Haven, Conn. (1999)); OPLS (Jorgensen, et al., J. Am. Chem. Soc. (1988), v 110, pp 1657ff; Jorgensen, et al., J. Am. Chem. Soc. (1990), v 112, pp 4768ff); UNRES (United Residue Forcefield; Liwo, et al., Protein Science (1993), v 2, pp1697-1714; Liwo, et al., Protein Science (1993), v 2, pp1715-1731; Liwo, et al., J. Comp. Chem. (1997), v 18, pp849-873; Liwo, et al., J. Comp. Chem. (1997), v 18, pp874-884; Liwo, et al., J. Comp. Chem. (1998), v 19, pp259-276; Forcefield for Protein Structure Prediction (Liwo, et al., Proc. Natl. Acad. Sci. USA (1999), v 96, pp5482-5485); ECEPP/3 (Liwo et al., J Protein Chem 1994 May;13(4):375-80); AMBER 1.1 force field (Weiner, et al., J. Am. Chem. Soc. v106, pp765-784); AMBER 3.0 force field (U. C. Singh et al., Proc. Natl. Acad. Sci. USA. 82:755-759); CHARMM and CHARMM22 (Brooks, et al., J. Comp. Chem. v4, pp 187-217); cvff3.0 (Dauber-Osguthorpe, et al., (1988) Proteins: Structure, Function and Genetics, v4, pp31-47); cff91 (Maple, et al., J. Comp. Chem. v15, 162-182); also, the DISCOVER (cvff and cff91) and AMBER forcefields are used in the INSIGHT molecular modeling package (Biosym/MSI, San Diego Calif.) and HARMM is used in the QUANTA molecular modeling package (Biosym/MSI, San Diego Calif.), all of which are expressly incorporated by reference.

Once the singles and doubles energies are calculated and stored, the next step of the computational processing may occur. As outlined in U.S. Pat. No. 6,269,312 and PCT Publication No. WO 98/47089, preferred embodiments utilize a Dead End Elimination (DEE) step, and preferably a Monte Carlo step.

PDA, viewed broadly, has three components that may be varied to alter the output (e.g. the library): the scoring functions used in the process; the filtering technique, and the sampling technique.

In a preferred embodiment, the scoring functions may be altered. In a preferred embodiment, the scoring functions outlined above may be biased or weighted in a variety of ways. For example, a bias towards or away from a reference sequence or family of sequences can be done; for example, a bias towards wild-type or homolog residues may be used. Similarly, the entire protein or a fragment of it may be biased; for example, the active site may be biased towards wild-type residues, or domain residues towards a particular desired physical property can be done. Furthermore, a bias towards or against increased energy can be generated. Additional scoring function biases include, but are not limited to applying electrostatic potential gradients or hydrophobicity gradients, adding a substrate or binding partner to the calculation, or biasing towards a desired charge or hydrophobicity.

In addition, in an alternative embodiment, there are a variety of additional scoring functions that may be used. Additional scoring functions include, but are not limited to torsional potentials, or residue pair potentials, or residue entropy potentials. Such additional scoring functions can be used alone, or as functions for processing the library after it is scored initially. For example, a variety of functions derived from data on binding of peptides to MHC (Major Histocompatibility Complex) can be used to rescore a library in order to eliminate proteins containing sequences which can potentially bind to MHC, i.e. potentially immunogenic sequences.

In a preferred embodiment, a variety of filtering techniques can be done, including, but not limited to, DEE and its related counterparts. Additional filtering techniques include, but are not limited to branch-and-bound techniques for finding optimal sequences (Gordon and Majo, Structure Fold. Des. 7:1089-98, 1999), and exhaustive enumeration of sequences. It should be noted however, that some techniques may also be done without any filtering techniques; for example, sampling techniques can be used to find good sequences, in the absence of filtering.

As will be appreciated by those in the art, once an optimized sequence or set of sequences is generated, (or again, these need not be optimized or ordered) a variety of sequence space sampling methods can be done, either in addition to the preferred Monte Carlo methods, or instead of a Monte Carlo search. That is, once a sequence or set of sequences is generated, preferred methods utilize sampling techniques to allow the generation of additional, related sequences for testing.

These sampling methods can include the use of amino acid substitutions, insertions or deletions, or recombinations of one or more sequences. As outlined herein, a preferred embodiment utilizes a Monte Carlo search, which is a series of biased, systematic, or random jumps. However, there are other sampling techniques that can be used, including Boltzman sampling, genetic algorithm techniques and simulated annealing. In addition, for all the sampling techniques, the kinds of jumps allowed can be altered (e.g. random jumps to random residues, biased jumps (to or away from wild-type, for example), jumps to biased residues (to or away from similar residues, for example), etc.). Jumps where multiple residue positions are coupled (two residues always change together, or never change together), jumps where whole sets of residues change to other sequences (e.g., recombination). Similarly, for all the sampling techniques, the acceptance criteria of whether a sampling jump is accepted can be altered, to allow broad searches at high temperature and narrow searches close to local optima at low temperatures. See Metropolis et al., J. Chem Phys v21, pp 1087, 1953, hereby expressly incorporated by reference.

In a preferred embodiment, particularly for longer proteins or proteins for which large samples are desired, the library sequences are used to create nucleic acids such as DNA which encode the member sequences and which can then be cloned into host cells, expressed and assayed, if desired. Thus, nucleic acids, and particularly DNA, can be made which encodes each member protein sequence using the methods described below. The choice of codons, suitable expression vectors and suitable host cells will vary depending on a number of factors, and can be easily optimized as needed.

4. Polynucleotide Construction

The rational diversity libraries described herein may be produced by a variety of methods available to one of skill in the art based on the disclosure herein which permit relatively inexpensive, rapid, and high fidelity construction of essentially any polynucleotide desired. For example, in one embodiment, diversity libraries may be constructed, for example, by hybridization based oligonucleotide assembly of overlapping complementary oligonucleotides (see e.g., Zhou et al. Nucleic Acids Research, 32: 5409-5417 (2004); Richmond et al. Nucleic Acids Research 32: 5011-5018 (2004); Tian et al. Nature 432: 1050-1054 (2004); and Carr et al. Nucleic Acids Research 32: e162 (2004)). For example, oligonucleotides having complementary, overlapping sequences may be synthesized on a chip and then eluted off. The oligonucleotides then self assemble based on hybridization of the complementary regions. This technique permits the production of long molecules of DNA having high fidelity.

In other embodiments, rational diversity libraries may be produced using PCR based assembly methods (including PAM or polymerase assembly multiplexing) and ligation based assembly methods (e.g., joining of nucleic acid segments having cohesive or blunt ends). In an exemplary embodiment, a plurality of polynucleotide constructs that form all or part of a rational diversity library may be assembled in a single reaction mixture. It should be understood that the compositions and methods described herein involving pools of nucleic acids are meant to encompass both support-bound and unbound nucleic acids, as well as combinations thereof.

Methods for performing assembly PCR are described, for example, in Kodumal et al. (2004) Proc. Natl. Acad. Sci. U.S.A. 101: 15573; Stemmer et al. (1995) Gene 164:49; Dillon et al. (1990) BioTechniques 9:298; Hayashi et al. (1994) BioTechniques 17:310; Chen et al. (1994) J. Am. Chem. Soc. 116:8799; Prodromou et al. (1992) Protein Eng. 5:827; U.S. Pat. Nos. 5,928,905 and 5,834,252; and U.S. Patent Application Publication Nos. 2003/0068643 and 2003/0186226.

In an exemplary embodiment, polymerase assembly multiplexing (PAM) may be used to produce the rational diversity libraries described herein (see e.g., Tian et al. (2004) Nature 432:1050; Zhou et al. (2004) Nucleic Acids Res. 32:5409; and Richmond et al. (2004) Nucleic Acids Res. 32:5011). Polymerase assembly multiplexing involves mixing sets of overlapping oligonucleotides and/or amplification primers under conditions that favor sequence-specific hybridization and chain extension by polymerase using the hybridizing strand as a template. The double stranded extension products may optionally be denatured and used for further rounds of assembly until a desired polynucleotide construct has been synthesized.

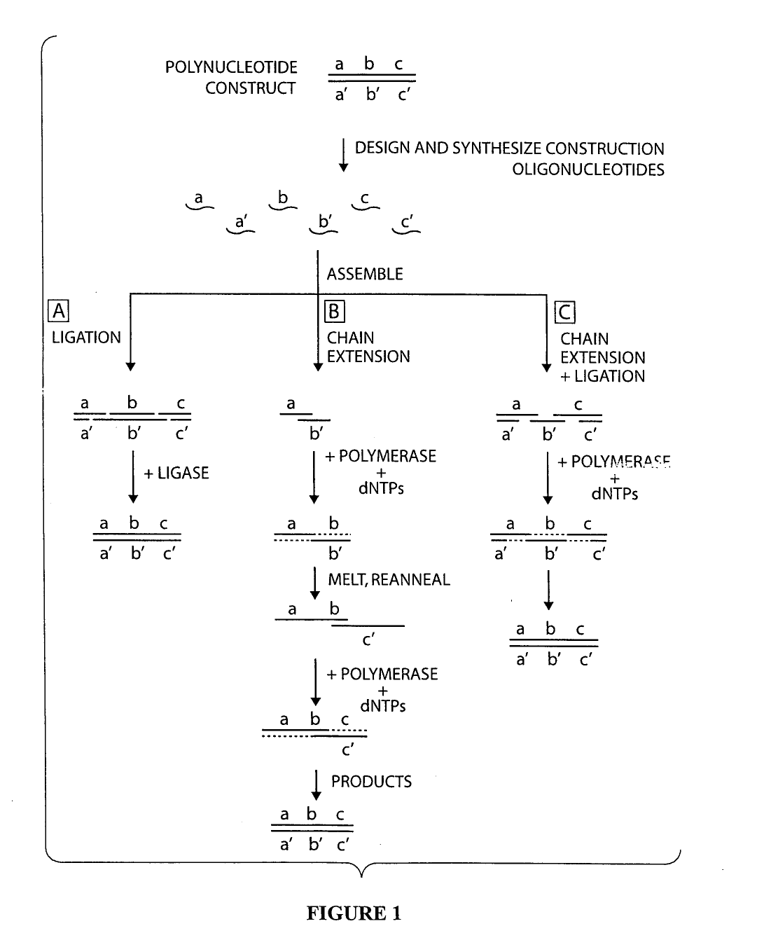

In certain embodiments, one or more members of a rational diversity library may be assembled by mixing together a plurality of shorter oligonucleotides having complementary overlapping regions that partially or completely comprise the sequence of the polynucleotide construct desired to be formed. For example, as illustrated in FIGS. 1B and 1C, the shorter oligonucleotides may form a partially double stranded nucleic acid that is assembled into a polynucleotide construct using chain extension, or a combination of chain extension and ligation, to fill in the gaps left between the shorter oligonucleotides. Alternatively, as illustrated in FIG. 1A, the shorter oligonucleotides may be designed so that upon assembly they abut one another and form a polynucleotide construct that only requires ligation between the shorter oligonucleotides to form the product (e.g., no gaps need to be filled in between the shorter oligonucleotides during the assembly process).

In one embodiment, polynucleotides suitable for construction of a rational diversity library may be produced, for example, using a nucleic acid array for the direct fabrication of DNA or other nucleic acid molecules of any desired sequence and of indefinite length. Sections or segments of the desired nucleic acid molecule are fabricated on an array, such as by way of a parallel nucleic acid synthesis process using an array synthesizer instrument. After the synthesis of the segments, the segments are assembled to make the desired molecule. In essence the technique permits the quick easy and direct synthesis of nucleic acid molecules for any purpose in a simple and quick synthesis process.

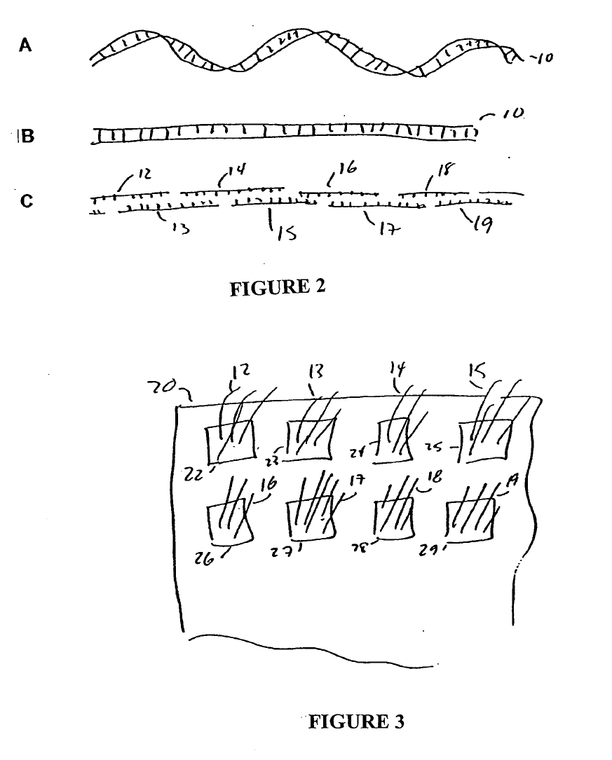

An illustration of the direct fabrication of a relatively simple DNA molecule is described in the figures. In FIG. 2, at 10, a double stranded DNA molecule of known sequence is illustrated. That same molecule is illustrated in both the familiar double helix shape in FIG. 2A, as well as in an untwisted double stranded linear shape shown in FIG. 2B. Assume, for purposes of this illustration, that the DNA molecule is broken up into a series of overlapping single smaller stranded DNA molecule segments, indicated by the reference numerals 12 through 19 in FIG. 2C. The even numbered segments are on one strand of the DNA molecule, while the odd numbered segments form the opposing complementary strand of the DNA molecule. The single stranded molecule segments can be of any reasonable length, but can be conveniently all of the same length which, for purposes of this example, might be 100 base pairs in length. Since the sequence of the molecule 10 of FIG. 2A is known, the sequence of the smaller DNA segments 12 through 19 can be defined simply be breaking the larger sequence into overlapping sequences each of, e.g., 75 to 100 base pairs.

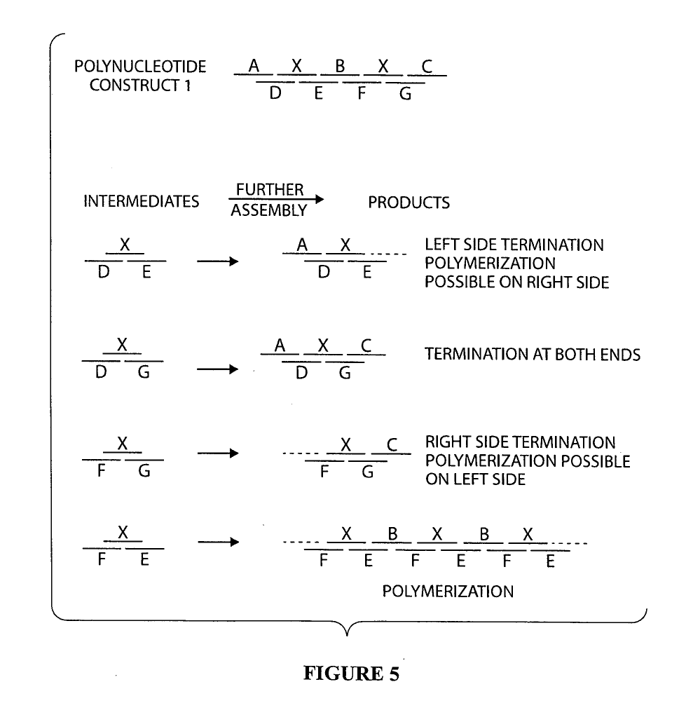

The information about the sequence of the segments 12-19 is then used to construct a new totally fabricated DNA molecule. This process is initiated by constructing a microarray of single stranded DNA segments on a common substrate. This process is illustrated in FIG. 3. Each of the single stranded segments 12 through 19 is constructed in a single cell, or feature, of a DNA microarray indicated at 20. Each of the DNA segments is fabricated in situ in a corresponding feature indicated by reference numbers 22 through 29. Such a microarray is preferably constructed using a maskless array synthesizer (MAS), as for example of the type described in published PCT patent application WO99/42813 and in corresponding U.S. Pat. No. 6,375,903, the disclosure of each of which is herein incorporated by reference. Other examples are known of maskless instruments which can fabricate a custom DNA microarray in which each of the features in the array has a single stranded DNA molecule of desired sequence. The preferred type of instrument is the type shown in FIG. 5 of U.S. Pat. No. 6,375,903, based on the use of reflective optics. It is a desirable and useful advantage of this type of maskless array synthesizer in that the selection of the DNA sequences of the single stranded DNA segments is entirely under software control. Since the entire process of microarray synthesis can be accomplished in only a few hours, and since suitable software permits the desired DNA sequences to be altered at will, this class of device makes it possible to fabricate microarrays including DNA segments of different sequence every day or even multiple times per day on one instrument. The differences in DNA sequence of the DNA segments in the microarray can also be slight or dramatic, it makes no different to the process. The usual use of such microarrays is to perform hybridization test on biological samples to test for the presence or absence of defined nucleic acids in the biological samples. Here, a much different use for the microarray is contemplated.

The MAS instrument may be used in the form it would normally be used to make microarrays for hybridization experiments, but it may also be adapted to have features specifically adapted for this application. For example, it may be desirable to substitute a coherent light source, i.e. a laser, for the light source shown in FIG. 5 of the above-mentioned U.S. Pat. No. 6,375,903. If a laser is used as the light source, a beam expanded and scatter plate may be used after the laser to transform the narrow light beam from the laser into a broader light source to illuminate the micromirror arrays used in the maskless array synthesizer. It is also envisioned that changes may be made to the flow cell in which the microarray is synthesized. In particular, it is envisioned that the flow cell can be compartmentalized, with linear rows of array elements being in fluid communication with each other by a common fluid channel, but each channel being separated from adjacent channels associated with neighboring rows of array elements. During microarray synthesis, the channels all receive the same fluids at the same time. After the DNA segments are separated from the substrate, the channels serve to permit the DNA segments from the row of array elements to congregate with each other an begin to self-assemble by hybridization. This alternative will also be discussed further below.

Once the fabrication of the DNA microarray is completed, the single stranded DNA molecule segments on the microarray are then freed or eluted from the substrate on which they were constructed. The particular method used to free the single stranded DNA segments is not critical, several techniques being possible. The DNA segment detachment method most preferred is a method which will be referred to here as the safety-catch method. Under the safety-catch approach, the initial starting material for the DNA strand construction in the microarray is attached to the substrate using a linker that is stable under the conditions required for DNA strand synthesis in the MAS instrument conditions, but which can be rendered labile by appropriate chemical treatment. After array synthesis, the linker is first rendered labile and then cleaved to release the single stranded DNA segments. The preferred method of detachment for this approach is cleavage by light degradation of a photo-labile attachment group.

The single stranded DNA molecules are suspended in a solution under conditions which favor the hybridization of single stranded DNA strands into double stranded DNA. Under these conditions, the single stranded DNA segments will automatically begin to assemble the desired larger complete DNA sequence. This occurs because, for example, the 3′ half of the DNA segment 12 will either preferentially or exclusively hybridize to the complementary half of the DNA segment 13. This is because of the complementary nature of the sequences on the 3′ half of the segment 12 and the sequence on the 5′ half of the segment 13. The half of the segment 13 that did not hybridize to the segment 12 will then, in turn, hybridize to the 3′ half of the segment 14. This process will continue spontaneously for all of the segments freed from the microarray substrate. By this process, a DNA assembly similar to that indicated in FIG. 2C is created. By joining the aligned single stranded DNA molecules to each other, as can be done with a DNA ligase, the DNA molecule 10 of FIG. 2A is completed. The number of copies of the molecule created will be proportional to the number of identical segments synthesized in each of the features in the microarray 20. It may also be desirable to assist the assembly of the completed DNA molecule be performing one of a number of types of sub-assembly reactions. Several alternatives for such reactions are described below.

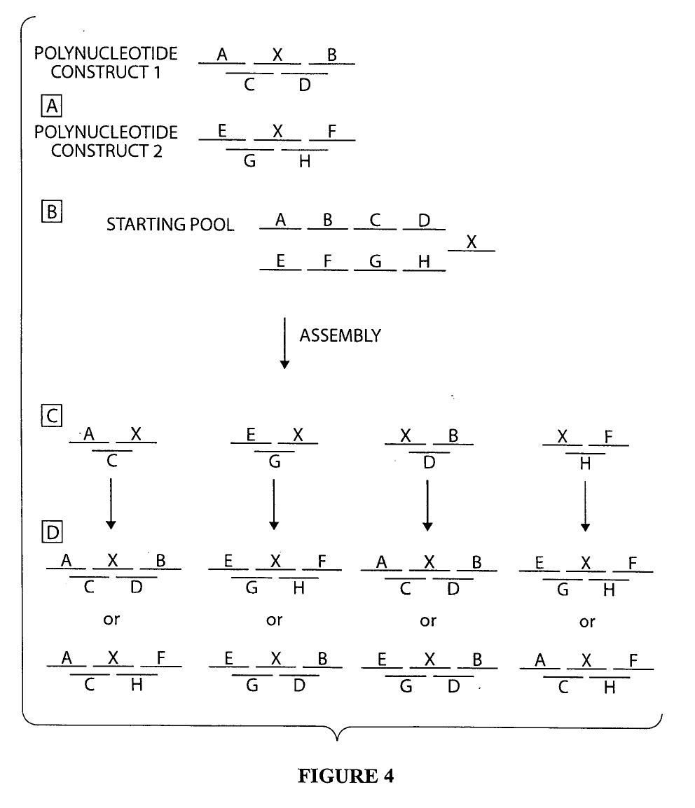

When conducting polymerase assembly multiplexing (PAM), homologous oligonucleotides can potentially act as crossover points leading to a mixture of full length products (FIGS. 4 and 5). Depending on the application, this can be a useful source of diversity, or a complication necessitating an additional separation step to obtain only the desired products. We have now discovered two strategies for accomplishing the selective separation of desired sequences from a mixture of crossover products: (1) selection by intermediate circularization and (2) selection by size. Both apply to PAM of polynucleotide constructs with one or more internal homologous regions.

In PAM (Tian et al., Nature 432: 1050-1054 (2004)), the order in which the oligonucleotide starting materials assemble to form polynucleotide constructs is defined by the mutual 5′ and 3′ complementarities of the oligonucleotides (Mullis et al., Cold Spring Harb. Symp. Quant. Biol. 51 pt 1: 263-273). The ends of each oligo can anneal to exactly one other oligo (except for the oligonucleotides at the end of a finished gene, which have a free end). This specificity of annealing ensures that only the desired full-length gene sequences will be assembled.

If there are sufficiently long regions of high homology among the genes to be synthesized in multiplexed format, however, this specificity can be lost. For example, when trying to synthesize two or more polynucleotide constructs that contain a highly homologous (or even identical) region X in a single pool, the common homologous region could lead to various assembled products in addition to the polynucleotide constructs of interest (see FIG. 4). This situation may arise when the homologous region X is at least as long as the construction oligonucleotide. This may occur, for example, when synthesizing polynucleotide constructs that encode closely related protein variants or proteins that share common domains. For example, as shown in FIG. 4, A, B, C, D, E, F, G, H and X denote non-homologous construction oligonucleotides. By design, the 5′ end of X can hybridize with both C and G, and the 3′ end of X can hybridize with both D and H. This does not present a complication if the two sets of oligonucleotides do not come into contact with each other (e.g., they are in separate pools). However, if synthesis is performed in a single well, four distinct full-length products will be formed (identified by top strand only): AXB, AXF, EXB, and EXF (see FIG. 4D). Therefore, when dealing with a homologous region, the number of different products that may be formed is s^{x+1}, where s is the number of homologous sequences and x is the number of internal crossover points.

Internal homologous regions (e.g., two regions contained in the same sequence which are highly homologous or identical) are a special case because they have the potential to lead to polymerization in PAM. As shown in FIG. 5, assembly of the AXBXC nucleic acid (represented by the top strand only) could lead to a family of products represented by AX(BX)ₙC, where n is any nonnegative integer. The number of products generated by this assembly is theoretically infinite.

In certain embodiments, it may be desirable to allow this type of combinatorial complexity to occur. For example, this crossover feature of PAM can be exploited to quickly and cheaply generate large combinatorial libraries for applications such as domain shuffling for protein design, creation of a library of RNAi molecules, creation of a library of aptamers, creation of library of Fab polypeptides, etc.

In other embodiments, it is desirable to minimize or eliminate combinatorial complexity and synthesize only a defined set of homologous sequences. This may be achieved by separately synthesizing genes containing homologous regions (to prevent crossover), for example, using separate pools that are mixed together in an ordered fashion to prevent crossover products. Alternatively, a variety of genes with homologous regions may be synthesized in a single pool and the undesired products may be removed using the separation techniques described below.

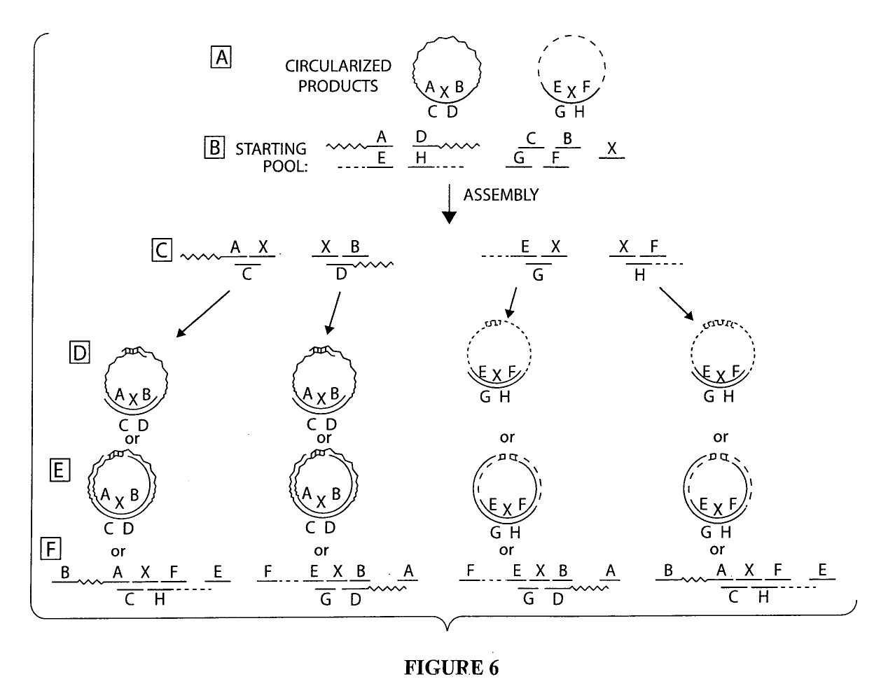

In one embodiment, undesired crossover products may be removed from a mixture of synthetic genes using a circle selection method. One embodiment of the circle selection method is illustrated in FIG. 6. The circle selection method takes advantage of the fact that circular single stranded DNA or double stranded DNA is exonuclease resistant. FIG. 6A illustrates two polynucleotide constructs that are desired to be constructed in a single pool (represented as a single strand for purposes of illustration). As shown in FIG. 6B, the terminal construction oligonucleotides are designed to form single stranded overhangs (which may optionally be formed by designing the construction oligonucleotides to contain an appropriate linker sequence) that allow the correct polynucleotide construct products to circularize, e.g., the complementary A/C oligonucleotides form a single stranded overhang that is complementary to a single stranded overhang formed by the complementary oligonucleotides B/D (represented by wavy lines) but are not complementary to a single stranded overhang formed by the F/H oligo pair (represented by dotted lines), etc. Therefore, only the correct products may circularize, while the incorrect crossover products (e.g., B-AXF-E and F-EXB-A) remain linear and may be degraded by an exonuclease leaving the circles intact (FIG. 6D-F). The flanking regions and circularizing segment are assembled, and then the homologous linker X is added to the mixture. The desired sequences then form circles (FIGS. 6D and 6E), while the crossover products form linear sequences (FIG. 6F). These crossover products can be selectively degraded using an exonuclease. Then, an appropriate enzyme (e.g., a restriction enzyme or uracil DNA glycosylase (UDG)) can be added to linearize the circles and/or remove the circularizing segment (linkers), leaving only the desired products, e.g., AXB and EXF (represented by top strand only). As shown in FIGS. 6D and 6E, the circularized products may be partially double stranded (FIG. 6D) or alternatively may be completely double stranded (FIG. 6E). It is also possible to convert partially double stranded circles to fully double stranded circles using a polymerase and dNTPs.

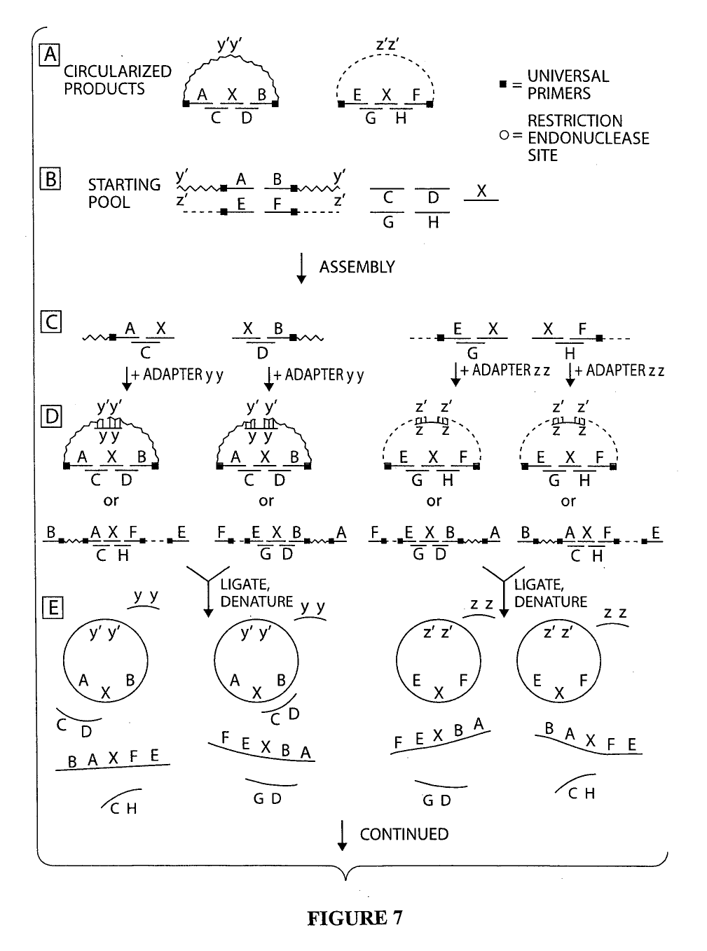

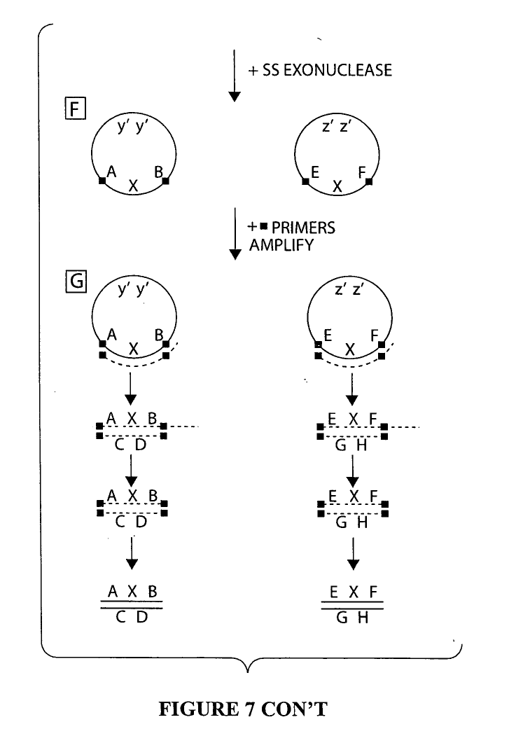

Another embodiment of the circle selection method is illustrated in FIG. 7. FIG. 7A shows the polynucleotide constructs that are desired to be synthesized in a single pool. FIG. 7B shows the construction oligonucleotides that define the polynucleotide constructs. The 5′ and 3′ most terminal construction oligonucleotides on the same strand contain flanking sequences that permit circularization of polynucleotide constructs that have been assembled in the proper order (e.g., oligonucleotides A and B, represented by wavy lines, and E and F, represented by dotted lines). After exposing the pool of polynucleotide constructs to hybridization conditions, linear sequences are added that are complementary to the flanking sequences of the terminal construction oligonucleotides. For example, as shown in FIGS. 7C and 7D, the adapter YY permits circularization of the AXB construct (e.g., by binding to the complementary Y′ regions) while the ZZ adapter permits circularization of the EXF construct (e.g., by binding to the complementary Z′ regions). However, incorrect crossover products (e.g., B-AXF-E and F-EXB-A) would have a combination of Y′ and Z′ complementary regions and therefore would not circularize upon exposure to the YY or ZZ adaptor oligonucleotides. The assembled constructs may then be ligated to form a covalently closed, partially single stranded circles and incorrect linear cross-over products (FIG. 7E). The constructs may then be denatured and subjected to a process to separate circles from linear nucleic acid strands (FIG. 7E-7F). This may be accomplished, for example, using a size separation method (e.g., circles will migrate through a PAGE gel faster than linear products) or using a single stranded exonuclease to digest the linear strands while leaving the circles intact. The correct assembly products may then be produced by amplifying the appropriate region of the circular product using primers that bind to a region flanking the AXB and EXF products (FIG. 7G). It should be understood that the adapter oligonucleotides are represented by YY and ZZ merely for purposes of illustration. The adapter oligonucleotides may be any combination of sequences that is complementary to the appropriate pair of construction oligonucleotides (e.g., the sequence complementary to a region of the 5′ construction oligonucleotide need not be the same as the sequence complementary to a region of the 3′ construction oligonucleotide).

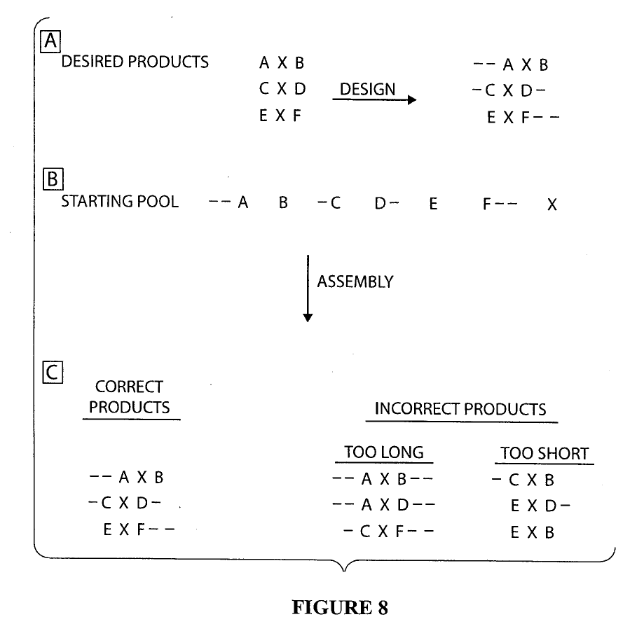

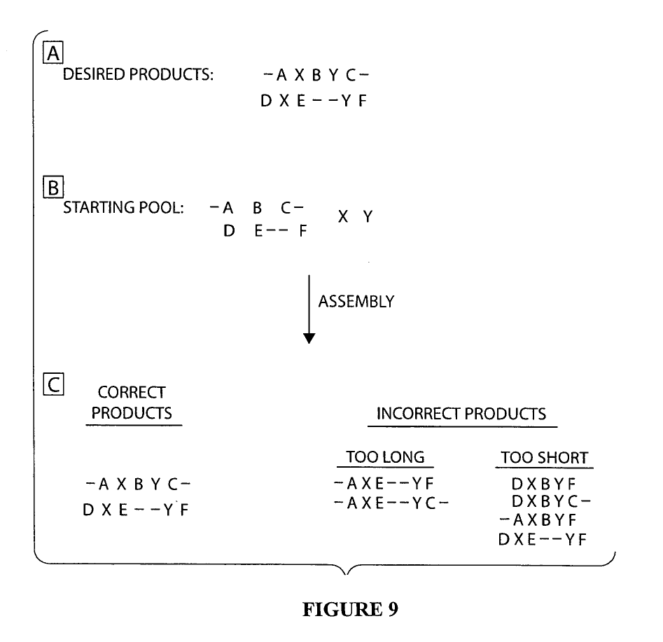

In another embodiment, undesired crossover products may be removed from a mixture of synthetic polynucleotide constructs using the size selection method which is illustrated in FIGS. 8 and 9. The size selection method takes advantage of the fact that the mobility of double stranded DNA is a function of its size, and thus DNA of different lengths can be separated, for example, via gel or column chromatography. In this embodiment, the initial polynucleotide constructs are designed such that the desired products have different lengths than all of the crossover products (see e.g., FIGS. 8A and 9A). For example, in one embodiment, the oligonucleotides are designed such that all of the desired products are about the same size, and any crossover products have significantly different sizes. This may be accomplished by designing the construction oligonucleotides such that the crossover point is in a different position in each of the target sequences. For example, as illustrated in FIG. 8, if the desired sequences are AXB, CXD, and EXF, and the A, B, C, C, E, F, and X are all approximately the same length, the sequences can be “padded” (e.g., the addition of extra bases or series of bases, represented as dashes) (FIG. 8B) to yield desired products having the same length, e.g., --AXB, -CXD-, and EXF--, and undesired crossover products having different lengths, e.g., --AXF--, --AXD-, -CXF--, -CXB, EXD-, or EXB (FIG. 8C). The polynucleotide constructs can be assembled in multiplexed format and the desired products separated from the crossover products by size selection. The padding units can then be removed using a restriction enzyme or UDG. In certain embodiments, such size selection techniques may be achieved merely through careful design of the construction oligonucleotides without the need to pad the oligonucleotides, e.g., the A, B, C, D, E, F, and X are naturally different sizes and will permit the distinction between correct vs. incorrect products.

The degree of difference in length needed to distinguish the products may be determined based on the separation method to be used. For example, if the size separation will be performed by gel electrophoresis, then a separation resolution and size differential of about +/−5-10% of the full nucleic acid sequence may be reasonable.

In another embodiment, if an internal region of DNA with known markers can be selectively excised, a single size selection could be used on sequences with more than one region of homology. This embodiment is illustrated in FIG. 9 for products AXBYC and DXEYF which may be synthesized in a single pool, for example, as -AXBYC- and DXE--YF (FIG. 9A) using the construction oligonucleotides shown in FIG. 9B. Of the 8 possible products (FIG. 9C), the 2 desired products each contain 2 units of padding (“−”), while the 6 crossover products at X or Y contain either 0, 1, 3, or 4 units of padding (FIG. 9C). The regions of internal padding may then be excised, for example, using a restriction endonuclease (e.g. a type IIS restriction endonuclease). The fragments may then be exposed to hybridization and ligation conditions to form the correct, unpadded construct.

In another embodiment, when multiple internal homologous regions are present, separate assembly and separation steps may be performed for each homologous region. The resulting gene fragments will then be unique and can be assembled via PAM. This is a “linear” strategy which scales in complexity as the number of homologous regions. As the molecule length grows, conventional methods of error-reduction become prohibitively cumbersome and costly. Set forth below are tools for dramatically reducing errors in large-scale gene synthesis.

In other embodiments, multiplex synthesis of sequences containing homologous regions may be achieved by careful design of the construction oligonucleotides. For example, the construction oligonucleotides may be codon remapped to reduce the level of homology while still maintaining or minimally changing any polypeptide sequence encoded by the nucleic acid. Additionally, the areas of complementarity between two or more construction oligonucleotides may be carefully chosen to reduce the level of homology in undesired regions of hybridization (see e.g., PCT Publication WO 00/43942). Methods for oligonucleotide design and codon remapping may be facilitated through the aid of computer design using, for example, DNAWorks (supra), Gene2Oligo (supra), or the implementation methods and systems discussed further below.

In another embodiment, methods for producing rational diversity libraries wherein members of the libray comprise two or more regions of self-homology are provided. The methods involve utilizing construction oligonucleotides that do not terminate within the regions of self-homology, e.g., one or more construction oligonucleotides span one or more regions of self-homology. When a polynucleotide construct comprises regions of self-homology that are large (e.g., a region of self-homology comprising more than about 100, 200, 500, or more base pairs), then the assembly procedure may comprise assembly of the different portions of the polynucleotide construct in separate pools. For example, a first portion of the polynucleotide construct comprising a first region of self-homology may be assembled in pool A and a second portion of the polynucleotide construct comprising a second region of self-homology may be assembled in pool B. The first and second regions of self-homology share homology with each other but do not share any substantial homology with other portions of the polynucleotide construct to be assembled in the same pool. After assembling the first and second portions of the polynucleotide construct in separate pools, the pools may be mixed to form the full length product, for example, by ligation, chain extension, or a combination thereof. If the polynucleotide construct contains a region of self-homology at one or both ends of the polynucleotide construct, non-homologous flanking sequences may be appended onto the end of the sequence so that construction oligonucleotides may be designed that do not terminate within a region of self-homology. The flanking sequences may be hypothetically appended onto one or both ends of the polynucleotide construct before designing the construction oligonucleotides or may be appended onto the ends of one or more construction oligonucleotides that correspond to the ends of the polynucleotide construct as appropriate.

In an exemplary embodiment, the biosynthetic, rational diversity libraries described herein may be constructed from oligonucleotides that have been codon remapped. The term “codon remapping” refers to modifying the codon content of a nucleic acid sequence without modifying the sequence of the polypeptide encoded by the nucleic acid. In certain embodiments, the term is meant to encompass “codon optimization” wherein the codon content of the nucleic acid sequence is modified to enhance expression in a particular cell type. In other embodiments, the term is meant to encompass “codon normalization” wherein the codon content of two or more nucleic acid sequences are modified to minimize any possible differences in protein expression that may arise due to the differences in codon usage between the sequences. In still other embodiments, the term is meant to encompass modifying the codon content of a nucleic acid sequence as a means to control the level of expression of a protein (e.g., either increases or decrease the level of expression). Codon remapping may be achieved by replacing at least one codon in the “wild-type sequence” with a different codon encoding the same amino acid that is used at a higher or lower frequency in a given cell type. For this embodiment, “wild-type” is meant to encompass sequences that have not been codon remapped whether they are true wild-type sequences or variant sequences designed using the methods described herein.

In an exemplary embodiment, the invention is directed to a plurality of nucleic acid molecules in a biosynthetic library that are codon normalized and/or codon optimized. Libraries of codon normalized nucleic acids will facilitate screening and/or selection of desired protein variants by minimizing experimental differences arising from variations in the levels of polypeptide expression due to codon bias (e.g., differences in enzymatic activities, binding affinities, etc.). Libraries of codon optimized nucleic acids will facilitate screening and/or selection of desired protein variants by optimizing expression in a given host cell. In an exemplary embodiment, libraries may comprise nucleic acids that have been both codon normalized and codon optimized.

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, nucleic acid sequences can be tailored for optimal expression in a given organism based on codon optimization.

Given the large number of gene sequences available for a wide variety of animal, plant and microbial species, it is possible to calculate the relative frequencies of codon usage. Codon usage tables are readily available, for example, at the “Codon Usage Database” available on the world wide web at kazusa.orjp/codon/, and these tables can be adapted in a number of ways. See Nakamura, Y., et al. “Codon usage tabulated from the international DNA sequence databases: status for the year 2000” Nucl. Acids Res. 28:292 (2000). These tables use mRNA nomenclature, and so instead of thymine (T) which is found in DNA, the tables use uracil (U) which is found in RNA. The tables have been adapted so that frequencies are calculated for each amino acid, rather than for all 64 codons.

By utilizing these or similar tables, one of ordinary skill in the art can apply the frequencies to any given polypeptide sequence, and produce a nucleic acid fragment of a codon-remapped coding region which encodes the same polypeptide, but which uses codons more or less optimal for a given species.

Codon-remapped coding regions can be designed by various different methods. For example, codon optimization may be carried out using a method termed “uniform optimization” wherein a codon usage table is used to find the single most frequent codon used for any given amino acid, and that codon is used each time that particular amino acid appears in the polypeptide sequence. For example, in humans the most frequent leucine codon is CUG, which is used 41% of the time. Therefore, codon optimatization may be carried out by assigning the codon CUG for all leucine residues in a given amino acid.

In another method, termed “full-optimization,” the actual frequencies of the codons are distributed randomly throughout the coding region. Thus, using this method for optimization, if a hypothetical polypeptide sequence had 100 leucine residues and was to be optimized for expression in human cells, about 7, or 7% of the leucine codons would be UUA, about 13, or 13% of the leucine codons would be UUG, about 13, or 13% of the leucine codons would be CUU, about 20, or 20% of the leucine codons would be CUC, about 7, or 7% of the leucine codons would be CUA, and about 41, or 41% of the leucine codons would be CUG. These frequencies would be distributed randomly throughout the leucine codons in the coding region encoding the hypothetical polypeptide. As will be understood by those of ordinary skill in the art, the distribution of codons in the sequence can vary significantly using this method, however, the sequence always encodes the same polypeptide. Such methods may be adapted similarly adapted for other codon remapping techniques, including codon normalization.

Randomly assigning codons at an optimized frequency to encode a given polypeptide sequence, can be done manually by calculating codon frequencies for each amino acid, and then assigning the codons to the polypeptide sequence randomly. Additionally, various algorithms and computer software programs are readily available to those of ordinary skill in the art. For example, the “EditSeq” function in the Lasergene Package, available from DNAstar, Inc., Madison, Wis., the backtranslation function in the Vector NTI Suite, available from InforMax, Inc., Bethesda, Md., and the “backtranslate” function in the GCG—Wisconsin Package, available from Accelrys, Inc., San Diego, Calif. In addition, various resources are publicly available to codon-optimize coding region sequences. For example, the “backtranslation” function on the world wide web at entelechon.com/eng/backtranslation.html, the “backtranseq” function available on the world wide web at bioinfo.pbi.nrc.ca:-8090/EMBOSS/index.html. Constructing a rudimentary algorithm to assign codons based on a given frequency can also easily be accomplished with basic mathematical functions by one of ordinary skill in the art.

In other embodiments, methods for producing rational diversity libraries may involve one or more error reduction procedures. Error reduction procedures allow removal or correction of errors introduced into the nucleic acid molecules at various stages during the assembly process including during on-chip synthesis, PCR amplification, PCR assembly, etc., and help to ensure high fidelity synthesis of the desired library members. Such error reduction procedures permit the use of low-purity arrays, e.g., arrays having features of less than 10 percent purity with respect to any given nucleic acid sequence. The ability to correct sequence errors allows the use of such low purity arrays to produce a high fidelity library product.

In various embodiments, mismatch binding proteins can be used to control the errors generated during oligonucleotide synthesis, gene assembly, and the construction of nucleic acids of different sizes. (Though biological systems use this function when synthesizing DNA, it requires the presence of a template strand. For de novo synthesis, as employed by this technique, one is starting by definition without a template.)

When attempting to produce a desired DNA molecule, a mixture typically results containing some correct copies of the sequence, and some containing one or more errors. But if the synthetic oligonucleotides are annealed to their complementary strands of DNA (also synthesized), then a single error at that sequence position on one strand will give rise to a base mismatch, causing a distortion in the DNA duplex. These distortions can be recognized by a mismatch binding protein. (One example of such a protein is MutS from the bacterium Escherichia coli.) Once an error is recognized, a variety of possibilities exist for how to prevent the presence of that error in the final desired DNA sequence.

When using pairs of complementary DNA strands for error recognition, each strand in the pair may contain errors at some frequency, but when the strands are annealed together, the chance of errors occurring at a correlated location on both strands is very small, with an even smaller chance that such a correlation will produce a correctly matched Watson-Crick base pair (e.g. A-T, G-C). For example, in a pool of 50-mer oligonucleotides, with a per-base error rate of 1%, roughly 60% of the pool (0.9950) will have the correct sequence, and the remaining forty percent will have one or more errors (primarily one error per oligonucleotide) in random positions. The same would be true for a pool composed of the complementary 50-mer. After annealing the two pools, approximately 36% (0.62) of the DNA duplexes will have correct sequence on both strands, 48% (2×0.4×0.6) will have an error on one strand, and 16% (0.42) will have errors in both strands. Of this latter category, the chance of the errors being in the same location is only 2% (1/50) and the chance of these errors forming a Watson-Crick base pair is even less (1/3×1/50). These correlated mismatches, which would go undetected, then comprise 0.11% of the total pool of DNA duplexes (16×1/3×/50). Removal of all detectable mismatch-containing sequences would thus enrich the pool for error-free sequences (i.e. reduce the proportion of error-containing sequences) by a factor of roughly 200 (0.6/0.4 originally for the single strands vs. 0.36/0.0011 after mismatch detection and removal). Furthermore, the remaining oligonucleotides can then be dissociated and re-annealed, allowing the error-containing strands to partner with different complementary strands in the pool, producing different mismatch duplexes. These can also be detected and removed as above, allowing for further enrichment for the error-free duplexes. Multiple cycles of this process can in principle reduce errors to undetectable levels. Since each cycle of error control may also remove some of the error-free sequences (while still proportionately enriching the pool for error-free sequences), alternating cycles of error control and DNA amplification can be employed to maintain a large pool of molecules.

In one embodiment, the number of errors detected and corrected may be increased by melting and reannealing a pool of DNA duplexes prior to error correction. For example, if the DNA duplexes in question have been amplified by a technique such as the polymerase chain reaction (PCR) the synthesis of new (perfectly) complementary strands would mean that these errors are not immediately detectable as DNA mismatches. However, melting these duplexes and allowing the strands to re-associate with new (and random) complementary partners would generate duplexes in which most errors would be apparent as mismatches, as described above.

Many of the methods described below can be used together, applying error-reducing steps at multiple points along the way to produce a long nucleic acid molecule. Error reduction can be applied to the first oligonucleotide duplexes generated, then for example to intermediate 500-mers or 1000-mers, and then even to larger full length nucleic acid sequences of 10,000-mers or more. In an exemplary embodiment, the methods described herein may be used to produce the entire genome of an organism optionally incorporating specific modifications into the sequence at one or more desired locations.

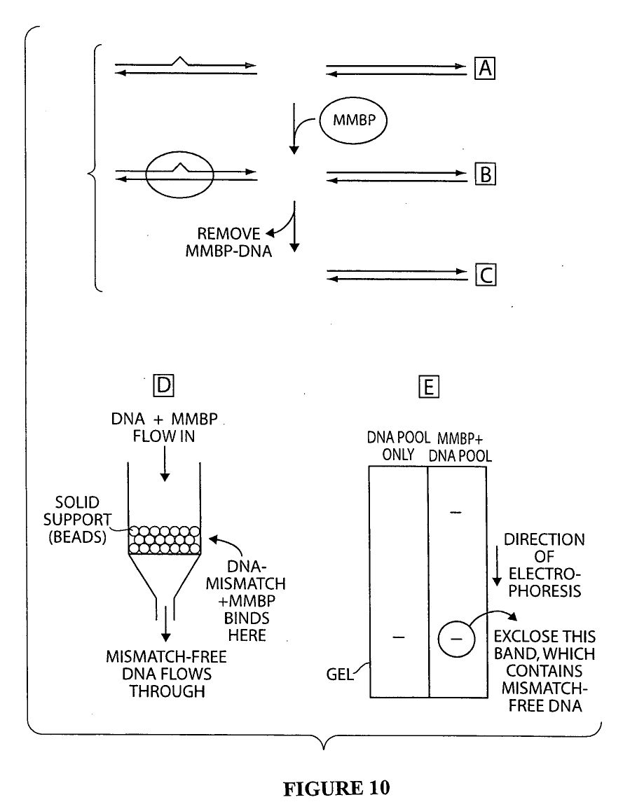

FIG. 10 illustrates an exemplary method for removing sequence errors using mismatch binding proteins. An error in a single strand of DNA causes a mismatch in a DNA duplex. A mismatch recognition protein (MMBP), such as a dimer of MutS, binds to this site on the DNA. As shown in FIG. 10A, a pool of DNA duplexes contains some duplexes with mismatches (left) and some which are error-free (right). The 3′-terminus of each DNA strand is indicated by an arrowhead. An error giving rise to a mismatch is shown as a raised triangular bump on the top left strand. As shown in FIG. 10B, a MMBP may be added which binds selectively to the site of the mismatch. The MMBP-bound DNA duplex may then be removed, leaving behind a pool which is dramatically enriched for error-free duplexes (FIG. 10C). In one embodiment, the DNA-bound protein provides a means to separate the error-containing DNA from the error-free copies (FIG. 10D). The protein-DNA complexes can be captured by affinity of the protein for a solid support functionalized, for example, with a specific antibody, immobilized nickel ions (protein is produced as a his-tag fusion), streptavidin (protein has been modified by the covalent addition of biotin) or other such mechanisms as are common to the art of protein purification. Alternatively, the protein-DNA complex is separated from the pool of error-free DNA sequences by a difference in mobility, for example, using a size-exclusion column chromatography or by electrophoresis (FIG. 10E). In this example, the electrophoretic mobility in a gel is altered upon MMBP binding: in the absence of MMBP all duplexes migrate together, but in the presence of MMBP, mismatch duplexes are retarded (upper band). The mismatch-free band (lower) is then excised and extracted.

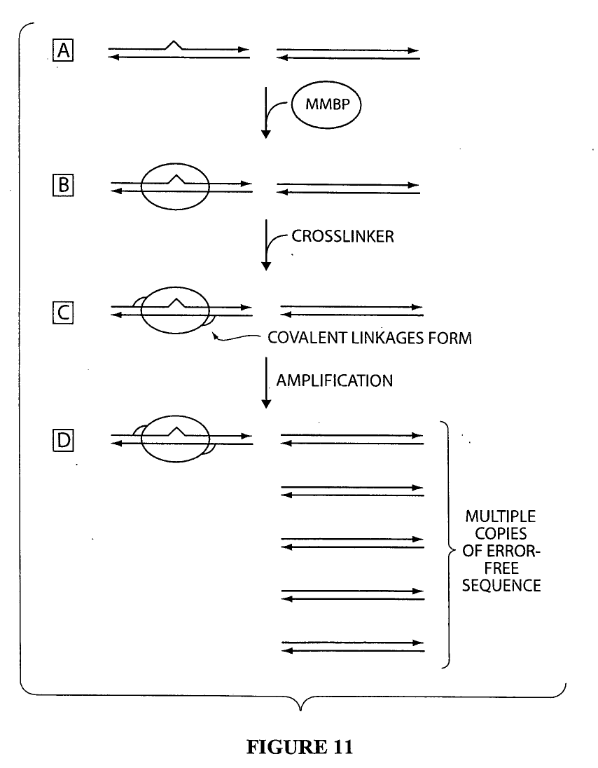

FIG. 11 illustrates an exemplary method for neutralizing sequence errors using mismatch recognition proteins. In this embodiment, the error-containing DNA sequence is not removed from the pool of DNA products. Rather, it becomes irreversibly complexed with a mismatch recognition protein by the action of a chemical crosslinking agent (for example, dimethyl suberimidate, DMS), or of another protein (such as MutL). The pool of DNA sequences is then amplified (such as by the polymerase chain reaction, PCR), but those containing errors are blocked from amplification, and quickly become outnumbered by the increasing error-free sequences. FIG. 11A illustrates an exemplary pool of DNA duplexes containing some duplexes with mismatches (left) and some which are error-free (right). A MMBP may be used to bind selectively to the DNA duplexes containing mismatches (FIG. 11B). The MMBP may be irreversibly attached at the site of the mismatch upon application of a crosslinking agent (FIG. 11C). In the presence of the covalently linked MMBP, amplification of the pool of DNA duplexes produces more copies of the error-free duplexes (FIG. 11D). The MMBP-mismatch DNA complex is unable to participate in amplification because the bound protein prevents the two strands of the duplex from dissociating. For long DNA duplexes, the regions outside the MMBP-bound site may be able to partially dissociate and participate in partial amplification of those (error-free) regions.

As increasingly longer sequences of DNA are generated, the fraction of sequences which are completely error-free diminishes. At some length, it becomes likely that there will be no molecule in the entire pool which contains a completely correct sequence. Thus, for the generation of extremely long segments of DNA, it can be useful to produce smaller units first which can be subjected to the above error control approaches. Then these segments can be combined to yield the larger full length product. However, if errors in these extremely long sequences can be corrected locally, without removing or neutralizing the entire long DNA duplex, then the more complex stepwise assembly process can be avoided.

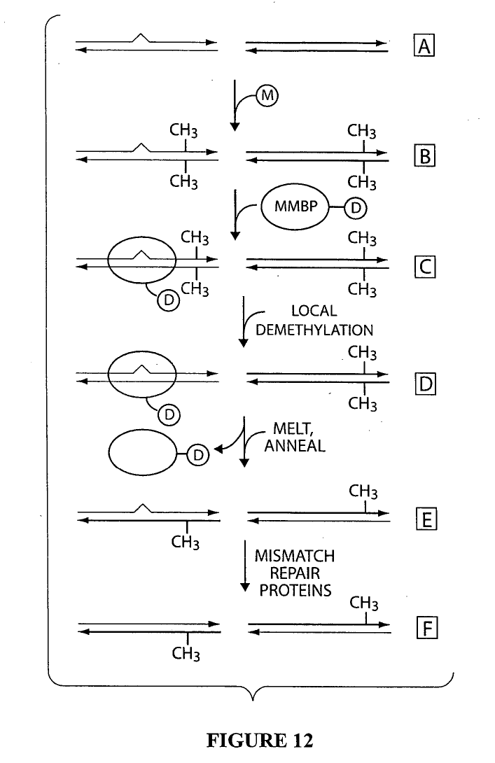

Many biological DNA repair mechanisms rely on recognizing the site of a mutation (error) and then using a template strand (most likely error-free) to replace the incorrect sequence. In the de novo production of DNA sequences, this process is complicated by the difficulty of determining which strand contains the error and which should be used as the template. In this invention, the solutions to this problem rely on using the pool of other sequences in the mixture to provide the template for correction. These methods can be very robust: even if every strand of DNA contains one or more errors, as long as the majority of strands have the correct sequence at each position (expected because the positions of errors are generally not correlated between strands), there is a high likelihood that a given error will be replaced with the correct sequence. FIGS. 12, 13, 14, and 15 present exemplary procedures for performing this sort of local error correction.

FIG. 12 illustrates an exemplary method for carrying out strand-specific error correction. In replicating organisms, enzyme-mediated DNA methylation is often used to identify the template (parent) DNA strand. The newly synthesized (daughter) strand is at first unmethylated. When a mismatch is detected, the hemimethylated state of the duplex DNA is used to direct the mismatch repair system to make a correction to the daughter strand only. However, in the de novo synthesis of a pair of complementary DNA strands, both strands are unmethylated, and the repair system has no intrinsic basis for choosing which strand to correct. In this aspect of the invention, methylation and site-specific demethylation are employed to produce DNA strands that are selectively hemi-methylated. A methylase, such as the Dam methylase of E. coli, is used to uniformly methylate all potential target sites on each strand. The DNA strands are then dissociated, and allowed to re-anneal with new partner strands. A new protein is applied, a fusion of a mismatch binding protein (MMBP) with a demethylase. This fusion protein binds only to the mismatch, and the proximity of the demethylase removes methyl groups from either strand, but only near the site of the mismatch. A subsequent cycle of dissociation and annealing allows the (demethylated) error-containing strand to associate with a (methylated) strand which is error-free in this region of its sequence. (This should be true for the majority of the strands, since the locations of errors on complementary strands are not correlated.) The hemi-methylated DNA duplex now contains all the information needed to direct the repair of the error, employing the components of a DNA mismatch repair system, such as that of E. coli, which employs MutS, MutL, MutH, and DNA polymerase proteins for this purpose. The process can be repeated multiple times to ensure all errors are corrected.

FIG. 12A shows two DNA duplexes that are identical except for a single base error in the top left strand, giving rise to a mismatch. The strands of the right hand duplex are shown with thicker lines. Methylase (M) may then be used to uniformly methylates all possible sites on each DNA strand (FIG. 12B). The methylase is then removed, and a protein fusion is applied, containing both a mismatch binding protein (MMBP) and a demethylase (D) (FIG. 12C). The MMBP portion of the fusion protein binds to the site of the mismatch thus localizing the fusion protein to the site of the mismatch. The demethylase portion of the fusion protein may then act to specifically remove methyl groups from both strands in the vicinity of the mismatch (FIG. 12D). The MMBP-D protein fusion may then be removed, and the DNA duplexes may be allowed to dissociated and re-associate with new partner strands (FIG. 12E). The error-containing strand will most likely re-associate with a complementary strand which a) does not contain a complementary error at that site; and b) is methylated near the site of the mismatch. This new duplex now mimics the natural substrate for DNA mismatch repair systems. The components of a mismatch repair system (such as E. coli MutS, MutL, MutH, and DNA polymerase) may then be used to remove bases in the error-containing strand (including the error), and uses the opposing (error-free) strand as a template for synthesizing the replacement, leaving a corrected strand (FIG. 12F).

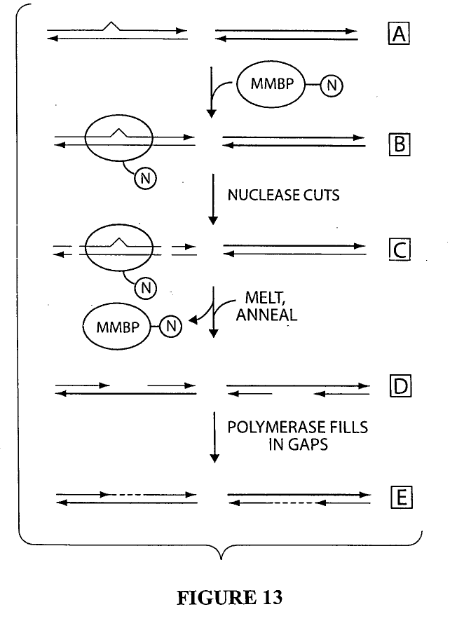

FIG. 13 illustrates an exemplary method for local removal of DNA on both strands at the site of a mismatch. Various proteins can be used to create a break in both DNA strands near an error. For example, an MMBP fusion to a non-specific nuclease (such as DNAseI) can direct the action of the nuclease (N) to the mismatch site, cleaving both strands. Once the break is generated, homologous recombination can be employed to use other strands (most of which will be error-free at this site) as template to replace the excised DNA. For example, the RecA protein can be used to facilitate single strand invasion, and early step in homologous recombination. Alternatively, a polymerase can be employed to allow broken strands to reassociate with new full-length partner strands, synthesizing new DNA to replace the error. For example, FIG. 13A shows two DNA duplexes that identical except that one contains a single base error as in FIG. 13A. In one embodiment, a protein, such as a fusion of a MMBP with a nuclease (N), may be added and will bind at the site of the mismatch (FIG. 13B). Alternatively, a nuclease with specificity for single-stranded DNA can be employed, using elevated temperatures to favor local melting of the DNA duplex at the site of the mismatch. (In the absence of a mismatch, a perfect DNA duplex will be less likely to melt.) An endonuclease, such as that of the MMBP-N fusion, may be used to make double-stranded breaks near the site of the mismatch (FIG. 13C). The MMBP-N complex is then removed, along with the bound short region of DNA duplex around the mismatch (FIG. 13D). Melting and re-annealing of partner strands produces some duplexes with single-stranded gaps. A DNA polymerase may then be used to fill in the gaps, producing DNA duplexes without the original error (FIG. 13E).

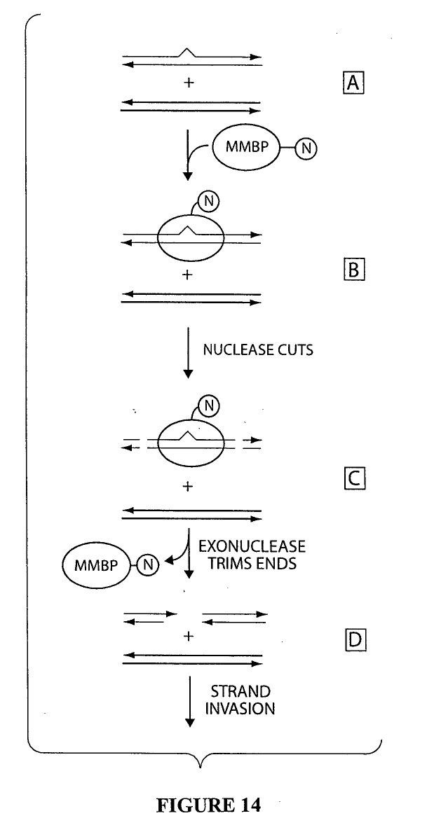

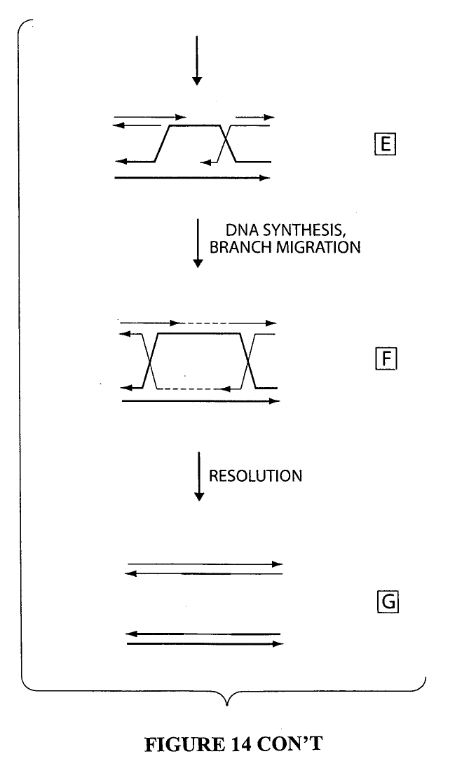

FIG. 14 illustrates a process similar to that of FIG. 13, however, in this embodiment, double-stranded gaps in DNA duplexes are repaired using the protein components of a recombination repair pathway. (Note that in this case no global melting and re-annealing of DNA strands is required, which can be preferable when dealing with especially large DNA molecules, such as genomic DNA.) For example, FIG. 14A shows two DNA duplexes (as in FIG. 13A), identical except that one contains a single base mismatch. As in FIG. 13B, a protein, such as a fusion of a MMBP with a nuclease (N), is added to bind at the site of the mismatch (FIG. 14B). As in FIG. 13C, an endonuclease, such as that of the MMBP-N fusion, may be used to make double-stranded breaks around the site of the mismatch (FIG. 14C). Protein components of a DNA repair pathway, such as the RecBCD complex, may then be employed to further digest the exposed ends of the double-stranded break, leaving 3′ overlaps (FIG. 14D). Subsequently, protein components of a DNA repair pathway, such as the RecA protein, are employed to facilitate single strand invasion of the intact DNA duplex, forming a Holliday junction (FIG. 14E). A DNA polymerase may then be used to synthesize new DNA, filling in the single-stranded gaps (FIG. 14F). Finally, protein components of a DNA repair pathway may be employed, such as the RuvC protein, to resolve the Holliday junction (FIG. 14G). The two resulting DNA duplexes do not contain the original error. Note that there can be more than one way to resolve such junctions, depending on migration of the branch points.

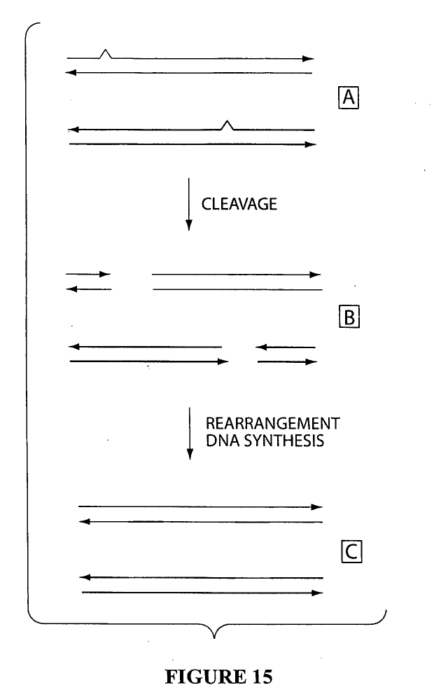

It is important to make clear that the methods described herein are capable of generating large error-free DNA sequences, even if none of the initial DNA products are error-free. FIG. 15 summarizes the effects of the methods of FIG. 13 (or equivalently, FIG. 14) applied to two DNA duplexes, each containing a single base (mismatch) error. For example, FIG. 15A illustrates two DNA duplexes, identical except for a single base mismatch in each, at different locations in the DNA sequence. Mismatch binding and localized nuclease activity are then used to generated double-stranded breaks which excise the errors (FIG. 15B). Recombination repair (as in FIG. 14) or melting and reassembly (as in FIG. 13) are employed to generate DNA duplexes where each excised error sequence has been replaced with newly synthesized sequence, each using the other DNA duplex as template (and unlikely to have an error in that same location) (FIG. 15C). Note that complete dissociation and re-annealing of the DNA duplexes is not necessary to generate the error-free products (if the methods shown in FIG. 14 are employed).

A simple way to reduce errors in long DNA molecules is to cleave both strands of the DNA backbone at multiple sites, such as with a site-specific endonuclease which generates short single stranded overhangs at the cleavage site. Of the resulting segments, some are expected to contain mismatches. These can be removed by the action and subsequent removal of a mismatch binding protein, as described in FIG. 10. The remaining pool of segments can be re-ligated into full length sequences. As with the approach of FIG. 14, this approach includes several advantages. 1) removal of an entire full length DNA duplex is not required to remove an error; 2) global dissociation and re-annealing of DNA duplexes is not necessary; 3) error-free DNA molecules can be constructed from a starting pool in which no one member is an error-free DNA molecule.

If the most common type of restriction endonuclease were employed for this approach, all DNA cleavage sites would result in identical overhangs. Thus the segments would associate and ligate in random order. However, use of a site-specific “outside cutter” endonuclease (such as HgaI, FokI, or BspMI) produces cleavage sites adjacent to (non-overlapping) the DNA recognition site. Thus each overhang would have sequence specific to that part of the DNA, distinct from that of the other sites. The re-association of these specifically complementary cohesive ends will then cause the segments to come together in the proper order. The cohesive ends generated can be up to five bases in length, allowing for up to 4⁵=1024 different combinations. Conceivably this many distinct restriction sites could be employed, though the need to avoid near matches between cohesive ends could lower this number.

The necessary restriction sites can be specifically included in the design of the sequence, or the random distribution of restriction sites within a desired sequence can be utilized (the recognition sequence of each endonuclease allows prediction of the typical distribution of fragments produced). Also, the target sequence can be analyzed for which choice of endonuclease produces the most ideal set of fragments.

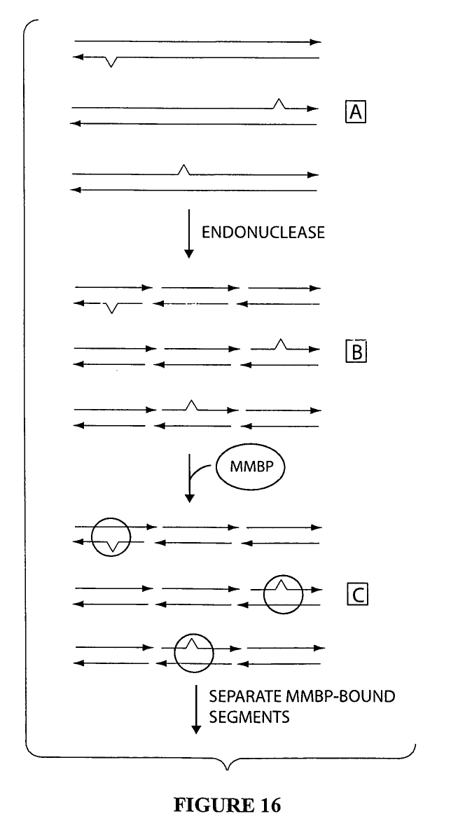

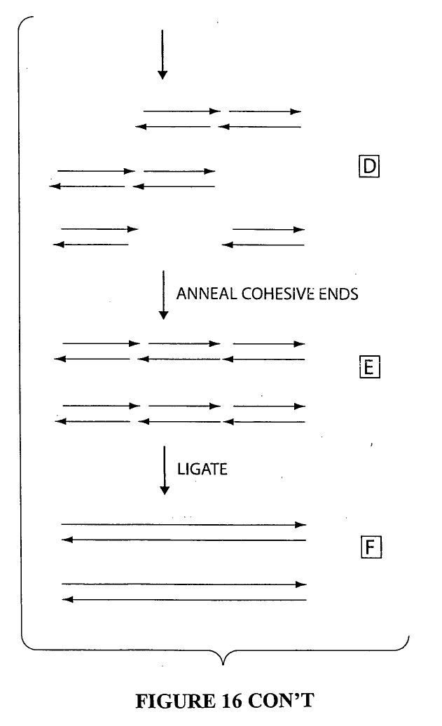

FIG. 16 shows an example of semi-selective removal of mismatch-containing segments. For example, FIG. 16A illustrates three DNA duplexes, each containing one error leading to a mismatch. The DNA is cut with a site-specific endonuclease, leaving double-stranded fragments with cohesive ends complementary to the adjacent segment (FIG. 16B). A MMBP is then applied, which binds to each fragment containing a mismatch (FIG. 16C). Fragments bound to MMBP are removed from the pool, as described in FIG. 10 (FIG. 16D). The cohesive ends of each fragment allow each DNA duplex to associate with the correct sequence-specific neighbor fragment (FIG. 16E). A ligase (such T4 DNA ligase) is employed to join the cohesive ends, producing full length DNA sequences (FIG. 16F). These DNA sequences can be error-free in spite of the fact that none of the original DNA duplexes was error-free. Incomplete ligation may leave some sequences which are less than full-length, which can be purified away on the basis of size.

The above approaches provide a major advantage over one of the conventional methods of removing errors, which employs sequencing first to find an error, and then relies on choosing specific error-free subsequences to “cut and paste” with endonuclease and ligase. In this embodiment, no sequencing or user choice is required in order to remove errors.

When complementary DNA strands are synthesized and allowed to anneal, both strands may contain errors, but the chance of errors occurring at the same base position in both sequences is extremely small, as discussed above. The above methods are useful for eliminating the majority case of uncorrelated errors which can be detected as DNA mismatches. In the rare case of complementary errors at identical positions on both strands (undetectable by the mismatch binding proteins), a subsequent cycle of duplex dissocation and random re-annealing with a different complementary strand (with a different distribution of error positions) remedies the problem. But in some applications it is desirable to not melt and re-anneal the DNA duplexes, such as in the case of genomic-length DNA strands. In such an embodiment, correlated errors may be removed using a different method. For example, though the initial population of correlated errors is expected to be low, amplification or other replication of the DNA sequences in a pool will ensure that each error is copied to produce a perfectly complementary strand which contains the complementary error. According to the invention that this approach does not require global dissociation and re-annealing of the DNA strands. Essentially, various forms of DNA damage and recombination are employed to allow single-stranded portions of the long DNA duplex to re-assort into different duplexes.

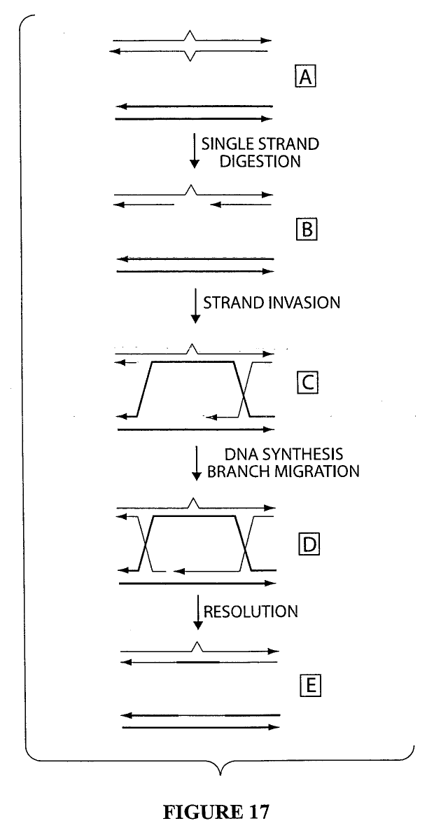

FIG. 17 shows a procedure for reducing correlated errors in synthesized DNA. FIG. 17A shows two DNA duplexes identical except for a single error in one strand. Non-specific nucleases may be used to generate short single-stranded gaps in random locations in the DNA duplexes in the pool (FIG. 17B). Shown here is the result of one of these gaps generated at the site of one of the correlated locations. Recombination-specific proteins such as RecA and RuvB are employed to mediate the formation of a four-stranded Holliday junction (FIG. 17C). DNA polymerase is employed to fill in the gap shown in the lower portion of the complex (FIG. 17D). Action of other recombination and/or repair proteins such as RuvC is employed to cleave the Holliday junction, resulting in two new DNA duplexes, containing some sequences which are hybrids of their progenitors (FIG. 17E). In the example shown, one of the error-containing regions has been eliminated. However, since the cutting, rearrangement, and replacement of strands employed in this method is intended to be random, it is expected that the total number of errors in the sequence will actually not change, simply that errors will be reassorted to different strands. Thus, pairs of errors correlated in one duplex will be reshuffled into separate duplexes, each with a single error. This random reassortment of strands will yield new duplexes containing mismatches which can be repaired using the mismatch repair proteins detailed above. Unique to this embodiment of the invention is the use of recombination to separate the correlated errors into different DNA duplexes.

The methods described above make possible the direct fabrication of DNA of any desired sequence. No longer do expression vectors have to be constructed from component parts by techniques of in vitro recombinant DNA. Instead, any desired DNA construct can be directly synthesized in total by direct synthesis in segments followed by spontaneous assembly into the completed molecule. The constructed DNA molecule does not have to be one that previously existed, it can be a totally novel construct to suit a particular purpose. It now becomes possible for one of skill in the art to design a desired DNA sequence or vector entirely in the computer, and then to directly synthesize the DNA vector artificially in a single operation.

It is envisioned that the process of direct DNA synthesis envisioned here will begin with a desired target DNA sequence, in the form of a computer file representing the target sequence that the user wants to build. A computer software program is used to determine the optimal way to subdivide the desired DNA construct into smaller DNA that can be used to build the larger target sequence. The software would be optimized for this purpose. For example, the target DNA construct should be subdivided into segments in such a manner so that the hybridizing half of each segment will hybridize well to a corresponding half segment, and not to any other half segment. If needed, changes to the sequence not affecting the ultimate functionality of the DNA may be required in some instances to ensure unique segments. This sort of optimization is preferable done by computer systems designed for this purpose.

After the DNA segments are constructed on the substrate of the microarray, the DNA segments must be separated from the microarray substrate. This can be done by any of a number of techniques, depending on the technique used to attach the DNA segments to the substrate in the first place. Described below is one technique based on base labile chemistry, adapted from techniques used to fabricate oligonucleotides on glass particles, but this is only one example among several possibilities. In essence, all that is required is that the attachment of the DNA segments to the substrate be cleaved by a technique that does not destroy the DNA molecules themselves.

This process may or may not make enough directly synthesized DNA as needed for a particular application. It is envisioned that more copies of the synthesized DNA can be made by any of the several ways in which other DNA constructs are cloned or replicated in quantity. An origin of replication can be built into circular DNA which would permit the rapid amplification of copies of the constructed DNA in a bacterial host. Linear DNA can be constructed with defined DNA primers at each end which can then be used to amplify many copies of the DNA construct by the PCR process.

5. Screening/Selection of Protein Variants

In exemplary embodiments, a variety of protein variants selected from the library of variants may be expressed and further screened to identify variants that exhibit one or more desired characteristics. Selection protocols are preferred over screening protocols because of their much more efficient thoughput rate, but both techniques can be used in an appropriate situation. Screening involves the assessment of a given construct for one or more properties of interest; selection involves retrieving or isolating species in a multispecies library having a particular property based on that property, e.g., panning, as is used in phage or ribosomal display. In one embodiment, the variants may be expressed using an in vitro transcription and/or translation system. In another embodiment, nucleic acids encoding the variants may be inserted into an expression vector and introduced into a cell for protein expression and screening or selection. Suitable methods for screening and selection for a biochemical characteristic of a variant include, for example, in vitro or in vivo assays for enzymatic activity or binding interactions (including protein/protein, protein/small molecule, etc.).

In one embodiment, using the nucleic acids of the present invention which encode library members, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the library protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the library protein, as will be appreciated by those in the art; for example, transcriptional and translational regulatory nucleic acid sequences from Bacillus are preferably used to express the library protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences include constitutive and inducible promoter sequences. The promoters may be either naturally occurring promoters, hybrid or synthetic promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors and appropriate selection and screening protocols are well known in the art and are described in e.g., Mansour et al., Cell, 51:503 (1988) and Murray, Gene Transfer and Expression Protocols, Methods in Molecular Biology, Vol. 7 (Clifton: Humana Press, 1991).

In addition, in a preferred embodiment, the expression vector contains a selection gene to allow the selection of transformed host cells containing the expression vector, and particularly in the case of mammalian cells, ensures the stability of the vector, since cells which do not contain the vector will generally die. Selection genes are well known in the art and will vary with the host cell used. By “selection gene” herein is meant any gene which encodes a gene product that confers resistance to a selection agent. Suitable selection agents include, but are not limited to, neomycin (or its analog G418), blasticidin S, histinidol D, bleomycin, puromycin, hygromycin B, and other drugs.

In a preferred embodiment, the expression vector contains a RNA splicing sequence upstream or downstream of the gene to be expressed in order to increase the level of gene expression. See Barret et al., Nucleic Acids Res. 1991; Groos et al., Mol. Cell. Biol. 1987; and Budiman et al., Mol. Cell. Biol. 1988.

A preferred expression vector system is a retroviral vector system such as is generally described in Mann et al., Cell, 33:153-9 (1993); Pear et al., Proc. Natl. Acad. Sci. U.S.A., 90(18):8392-6 (1993); Kitamura et al., Proc. Natl. Acad. Sci. U.S.A., 92:9146-50 (1995); Kinsella et al., Human Gene Therapy, 7:1405-13; Hofmann et al., Proc. Natl. Acad. Sci. U.S.A., 93:5185-90; Choate et al., Human Gene Therapy, 7:2247 (1996); PCT/US97/01019 and PCT/US97/01048, and references cited therein, all of which are hereby expressly incorporated by reference.

The library proteins of the present invention are produced by culturing a host cell transformed with nucleic acid, preferably an expression vector, containing nucleic acid encoding an library protein, under the appropriate conditions to induce or cause expression of the library protein. The conditions appropriate for library protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

As will be appreciated by those in the art, the type of cells used in the present invention can vary widely. Basically, a wide variety of appropriate host cells can be used, including yeast, bacteria, archaebacteria, fungi, and insect and animal cells, including mammalian cells. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, immortalized mammalian myeloid and lymphoid cell lines, Jurkat cells, mast cells and other endocrine and exocrine cells, and neuronal cells. See the ATCC cell line catalog, hereby expressly incorporated by reference. In addition, the expression of the secondary libraries in phage display systems, such as are well known in the art, are particularly preferred, especially when the secondary library comprises random peptides. In one embodiment, the cells may be genetically engineered, that is, contain exogeneous nucleic acid, for example, to contain target molecules.

In a preferred embodiment, the library proteins are expressed in mammalian cells. Any mammalian cells may be used, with mouse, rat, primate and human cells being particularly preferred, although as will be appreciated by those in the art, modifications of the system by pseudotyping allows all eukaryotic cells to be used, preferably higher eukaryotes. As is more fully described below, a screen will be set up such that the cells exhibit a selectable phenotype in the presence of a random library member. As is more fully described below, cell types implicated in a wide variety of disease conditions are particularly useful, so long as a suitable screen may be designed to allow the selection of cells that exhibit an altered phenotype as a consequence of the presence of a library member within the cell.

Accordingly, suitable mammalian cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog, hereby expressly incorporated by reference.

Mammalian expression systems are also known in the art, and include retroviral systems. A mammalian promoter is any DNA sequence capable of binding mammalian RNA polymerase and initiating the downstream (3′) transcription of a coding sequence for library protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase 11 to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, library proteins are expressed in bacterial systems. Bacterial expression systems are well known in the art.

A suitable bacterial promoter is any nucleic acid sequence capable of binding bacterial RNA polymerase and initiating the downstream (3′) transcription of the coding sequence of library protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the library protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E. coli, Streptococcus cremoris, and Streptococcus lividans, among others.

The bacterial expression vectors are transformed into bacterial host cells using techniques well known in the art, such as calcium chloride treatment, electroporation, and others.

In one embodiment, library proteins are produced in insect cells. Expression vectors for the transformation of insect cells, and in particular, baculovirus-based expression vectors, are well known in the art and are described e.g., in O'Reilly et al., Baculovirus Expression Vectors: A Laboratory Manual (New York: Oxford University Press, 1994).

In a preferred embodiment, library protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUPI gene, which allows yeast to grow in the presence of copper ions.

The library protein may also be made as a fusion protein, using techniques well known in the art. Thus, for example, for the creation of monoclonal antibodies, if the desired epitope is small, the library protein may be fused to a carrier protein to form an immunogen. Alternatively, the library protein may be made as a fusion protein to increase expression, or for other reasons. For example, when the library protein is a library peptide, the nucleic acid encoding the peptide may be linked to other nucleic acid for expression purposes. Similarly, other fusion partners may be used, such as targeting sequences which allow the localization of the library members into a subcellular or extracellular compartment of the cell, rescue sequences or purification tags which allow the purification or isolation of either the library protein or the nucleic acids encoding them; stability sequences, which confer stability or protection from degradation to the library protein or the nucleic acid encoding it, for example resistance to proteolytic degradation, or combinations of these, as well as linker sequences as needed.

Thus, suitable targeting sequences include, but are not limited to, binding sequences capable of causing binding of the expression product to a predetermined molecule or class of molecules while retaining bioactivity of the expression product, (for example by using enzyme inhibitor or substrate sequences to target a class of relevant enzymes); sequences signalling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane; and b) extracellular locations via a secretory signal. Particularly preferred is localization to either subcellular locations or to the outside of the cell via secretion.

In a preferred embodiment, the library member comprises a rescue sequence. A rescue sequence is a sequence which may be used to purify or isolate either the candidate agent or the nucleic acid encoding it. Thus, for example, peptide rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, and GST.

Alternatively, the rescue sequence may be a unique oligonucleotide sequence which serves as a probe target site to allow the quick and easy isolation of the retroviral construct, via PCR, related techniques, or hybridization.

In a preferred embodiment, the fusion partner is a stability sequence to confer stability to the library member or the nucleic acid encoding it. Thus, for example, peptides may be stabilized by the incorporation of glycines after the initiation methionine (MG or MGGO), for protection of the peptide to ubiquitination as per Varshavsky's N-End Rule, thus conferring long half-life in the cytoplasm. Similarly, two prolines at the C-terminus impart peptides that are largely resistant to carboxypeptidase action. The presence of two glycines prior to the prolines impart both flexibility and prevent structure initiating events in the di-proline to be propagated into the candidate peptide structure. Thus, preferred stability sequences are as follows: MG(X)ₙGGPP, where X is any amino acid and n is an integer of at least four.

In one embodiment, the library nucleic acids, proteins and antibodies of the invention are labeled. By “labeled” herein is meant that nucleic acids, proteins and antibodies of the invention have at least one element, isotope or chemical compound attached to enable the detection of nucleic acids, proteins and antibodies of the invention. In general, labels fall into three classes: a) isotopic labels, which may be radioactive or heavy isotopes; b) immune labels, which may be antibodies or antigens; and c) colored or fluorescent dyes. The labels may be incorporated into the compound at any position.

In a preferred embodiment, the library protein is purified or isolated after expression. Library proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the library protein may be purified using a standard anti-library antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the library protein. In some instances no purification will be necessary.

Once expressed and purified if necessary, the library proteins and nucleic acids are useful in a number of applications.

In general, the libraries are screened for biological activity. These screens will be based on the scaffold protein chosen, as is known in the art. Thus, any number of protein activities or attributes may be tested, including its binding to its known binding members (for example, its substrates, if it is an enzyme), activity profiles, stability profiles (pH, thermal, buffer conditions), substrate specificity, immunogenicity, toxicity, etc.

When random peptides are made, these may be used in a variety of ways to screen for activity. In a preferred embodiment, a first plurality of cells is screened. That is, the cells into which the library member nucleic acids are introduced are screened for an altered phenotype. Thus, in this embodiment, the effect of the library member is seen in the same cells in which it is made; i.e. an autocrine effect.

Thus, in one embodiment, the methods of the present invention comprise introducing a molecular library of library members into a plurality of cells, a cellular library. The plurality of cells is then screened, as is more fully outlined below, for a cell exhibiting an altered phenotype. The altered phenotype is due to the presence of a library member.

By “altered phenotype” or “changed physiology” or other grammatical equivalents herein is meant that the phenotype of the cell is altered in some way, preferably in some detectable and/or measurable way. As will be appreciated in the art, a strength of the present invention is the wide variety of cell types and potential phenotypic changes which may be tested using the present methods. Accordingly, any phenotypic change which may be observed, detected, or measured may be the basis of the screening methods herein. Suitable phenotypic changes include, but are not limited to: gross physical changes such as changes in cell morphology, cell growth, cell viability, adhesion to substrates or other cells, and cellular density; changes in the expression of one or more RNAs, proteins, lipids, hormones, cytokines, or other molecules; changes in the equilibrium state (i.e. half-life) or one or more RNAs, proteins, lipids, hormones, cytokines, or other molecules; changes in the localization of one or more RNAs, proteins, lipids, hormones, cytokines, or other molecules; changes in the bioactivity or specific activity of one or more RNAs, proteins, lipids, hormones, cytokines, receptors, or other molecules; changes in phosphorylation; changes in the secretion of ions, cytokines, hormones, growth factors, or other molecules; alterations in cellular membrane potentials, polarization, integrity or transport; changes in infectivity, susceptability, latency, adhesion, and uptake of viruses and bacterial pathogens; etc. By “capable of altering the phenotype” herein is meant that the library member can change the phenotype of the cell in some detectable and/or measurable way.

The altered phenotype may be detected in a wide variety of ways, and will generally depend and correspond to the phenotype that is being changed. Generally, the changed phenotype is detected using, for example: microscopic analysis of cell morphology; standard cell viability assays, including both increased cell death and increased cell viability, for example, cells that are now resistant to cell death via virus, bacteria, or bacterial or synthetic toxins; standard labeling assays such as fluorometric indicator assays for the presence or level of a particular cell or molecule, including FACS or other dye staining techniques; biochemical detection of the expression of target compounds after killing the cells; etc. In some cases, as is more fully described herein, the altered phenotype is detected in the cell in which the randomized nucleic acid was introduced; in other embodiments, the altered phenotype is detected in a second cell which is responding to some molecular signal from the first cell.

Thus, in a preferred embodiment, the invention provides biochips comprising libraries of variant proteins, with the library comprising at least about 100 different variants, with at least about 500 different variants being preferred, about 1000 different variants being particularly preferred and about 5000-10,000 being especially preferred.

In one embodiment, the candidate library is fully randomized, with no sequence preferences or constants at any position. In a preferred embodiment, the candidate library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

In a preferred embodiment, the bias is towards peptides or nucleic acids that interact with known classes of molecules. For example, when the candidate bioactive agent is a peptide, it is known that much of intracellular signaling is carried out via short regions of polypeptides interacting with other polypeptides through small peptide domains. For instance, a short region from the HIV-1 envelope cytoplasmic domain has been previously shown to block the action of cellular calmodulin. Regions of the Fas cytoplasmic domain, which shows homology to the mastoparan toxin from Wasps, can be limited to a short peptide region with death-inducing apoptotic or G protein inducing functions. Magainin, a natural peptide derived from Xenopus, can have potent anti-tumour and anti-microbial activity. Short peptide fragments of a protein kinase C isozyme (βPKC), have been shown to block nuclear translocation of βPKC in Xenopus oocytes following stimulation. And, short SH-3 target peptides have been used as psuedosubstrates for specific binding to SH-3 proteins. This is of course a short list of available peptides with biological activity, as the literature is dense in this area. Thus, there is much precedent for the potential of small peptides to have activity on intracellular signaling cascades. In addition, agonists and antagonists of any number of molecules may be used as the basis of biased randomization of candidate bioactive agents as well.

Thus, a number of molecules or protein domains are suitable as starting points for the generation of biased randomized candidate bioactive agents. A large number of small molecule domains are known, that confer a common function, structure or affinity. In addition, as is appreciated in the art, areas of weak amino acid homology may have strong structural homology. A number of these molecules, domains, and/or corresponding consensus sequences, are known, including, but are not limited to, SH-2 domains, SH-3 domains, Pleckstrin, death domains, protease cleavage/recognition sites, enzyme inhibitors, enzyme substrates, Traf, etc. Similarly, there are a number of known nucleic acid binding proteins containing domains suitable for use in the invention. For example, leucine zipper consensus sequences are known.

### INCORPORATION BY REFERENCE

All of the patents, publications and sequence database entries cited herein are hereby incorporated by reference. Also incorporated by reference are the following: U.S. Patent Application Publication Nos: 2004/0259146; 2004/0241701; 2003/0096307; 2004/0043430; 2003/0036854; 2004/0152872; and 2002/0177691.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for producing a protein having a desired characteristic comprising vi) applying an algorithm to a protein scaffold to generate a plurality of possible variants;
vii) screening the plurality of variants in silico to produce a rank ordered list of variants;
viii) generating nucleic acid molecules having predefined sequences that encode at least 10 of the variants wherein the nucleic acid molecules are generated by a method comprising:
a) providing a pool of oligonucleotides comprising partially overlapping sequences that define the sequence of each of said nucleic acid molecules that encode said variants;
b) incubating said pool of oligonucleotides under hybridization conditions and at least one of the following conditions: (i) ligation conditions, (2) chain extension conditions, or (iii) chain extension and ligation conditions, thereby forming nucleic acid constructs; and
c) separating constructs having said predefined sequences from constructs not having said predefined sequences, thereby forming the nucleic acid molecules that encode said variants; and
ix) causing expression of said nucleic acid molecules to produce said protein variants; and
x) screening the variants to identify variants having the desired characteristic.

2. The method of claim 1, wherein nucleic acids encoding at least 1000 of the variants are generated.

3. The method of claim 1, wherein nucleic acids encoding at least 10,000 of the variants are generated.

4. The method of claim 1, wherein the nucleic acids encoding the variants are at least 1000 bases in length.

5. The method of claim 1, wherein the nucleic acids encoding the variants are at least 5000 bases in length.

6. The method of claim 1, wherein the variants are produced in vitro.

7. The method of claim 1, wherein the nucleic acid molecules encoding the variants are prepared in a single pool.

8. The method of claim 1, wherein at least a portion of the sequence of one or more nucleic acids has been codon remapped to reduce the homology with at least one other nucleic acid.

9. The method of claim 1, wherein the oligonucleotides are synthesized on an array.

10. The method of claim 9, wherein the array comprises a solid support and a plurality of discrete features associated with said solid support, wherein each feature independently comprises a population of oligonucleotides collectively having a defined consensus sequence but in which no more than 10 percent of said oligonucleotides of said feature have the identical sequence.

11. The method of claim 1, wherein the method for generating the nucleic acid molecules further comprises an error reduction process.

12. The method of claim 1, wherein the nucleic acid molecules encoding the variants comprise sticky ends.

13. The method of claim 1, wherein one or more of the oligonucleotides that define the sequence of the nucleic acid molecules further comprise sequence tags such that a set of oligonucleotides that defines the sequence of a nucleic acid construct having a desired sequence has a distinguishable complement of sequence tags as compared to a set of oligonucleotides that defines the sequence of an incorrect product, and wherein nucleic acid constructs having a desired sequence are separated from incorrect crossover products based on size or electrophoretic mobility.

14. The method of claim 1, wherein a set of oligonucleotides that defines the sequence of a nucleic acid construct having a desired sequence forms sticky ends that permit circularization of the correctly formed product, and wherein correctly formed circularized products are separated from incorrectly formed linear products.

15. The method of claim 14, wherein the circularized products are separated from the linear products by digesting the linear products with an exonuclease.

16. The method of claim 1, wherein the nucleic acid molecules encoding the variants comprise vector sequences and sticky ends that permit circularization of the nucleic acid molecule to produce a circularized expression plasmid.

17. A biosynthetic library comprising a plurality of synthetic DNAs encoding a plurality of candidate proteins which can be selected or screened for species having a predetermined property or set of properties, the library comprising plural DNAs comprising regions of sequence homology and being assembled from chemically synthesized oligonucleotides.

18. A biosynthetic library comprising a plurality of synthetic DNAs encoding a plurality of candidate proteins which can be selected or screened for species having a predetermined property or set of properties, the library comprising plural DNAs chemically synthesized or assembled from chemically synthesized oligonucleotides and comprising reading frames of multiple said DNAs exploiting consistent codon usage patterns so as to promote similar expression levels in a selected expression system.

19. The library of claim 18, wherein said chemically synthesized oligonucleotides are synthesized in parallel.

20. The library of claim 18, wherein said DNAs are assembled in parallel from chemically synthesized oligonucleotides.
